(19) United States
(12) Patent Application Publication
(10) Pub. No.: US 2025/0312069 A1
(43) Pub. Date: Oct. 9, 2025

(54) BONE SEGMENT TRANSLATION SYSTEM

(71) Applicant: Blue Ocean Global Corp., Miami, FL (US)

(72) Inventors: Robert Scott Ludecker, Southold, NY (US); Ethan B. Ludecker, Charlottesville, VA (US); Michael H. Theodoulou, Bedford, NH (US); Alan B. Miller, Jamison, PA (US)

(21) Appl. No.: 18/663,849

(22) Filed: May 14, 2024

Related U.S. Application Data
(60) Provisional application No. 63/574,357, filed on Apr. 4, 2024.

(51) Int. Cl. A61B 17/66; A61B 17/64

(52) U.S. Cl. CPC A61B 17/66; A61B 17/6475

(57) ABSTRACT

A bone segment translation system comprises an external fixation element, a first block fixed to the external fixation element, a second block movably coupled to the first block, and a plurality of pins coupled to the second block. The plurality of pins are configured to be affixed to a bone segment of a bone of a subject.

### FIELD

The field relates generally to the translation of a bone segment and systems and methods of performing same.

### BACKGROUND

Diabetes mellitus affects hundreds of millions of people globally and is a leading cause of chronic disease. One of the major complications of diabetes mellitus is diabetic foot ulcers, which may result in complications such as gangrene and infection, sometimes leading to amputation. Chronic neuropathy may lead to insensitive and deformed feet, causing high pressure on some foot areas and eventual ulceration. In addition, peripheral artery disease can lead to ischemic ulceration.

Diabetic foot ulcers present multiple challenges as they can affect bones, nerves, vascular tissues, muscles, and skin. In some approaches to treat diabetic foot ulcers, treatments are designed to cause regeneration of multiple tissues. For example, translation osteogenesis has resulted in new bone growth, neovascularization and increased perfusion to bones and surrounding tissues. In some cases, translational movement of a leg bone segment promoted healing of diabetic foot ulcers by stimulating neovascularization and improving perfusion of the foot. However, devices and systems for effectively implementing bone segment translation are lacking and fail to deliver important nutrients through the endosteal cavity that flows through marrow sinusoids and nutrient arteries before exiting via numerous small vessels that ramify through the bone segment.

### SUMMARY

Illustrative embodiments provide a bone segment translation system comprising an external fixation device configured to cause translation of a bone segment transversely with respect to a first position.

According to an illustrative embodiment, a bone segment translation system comprises an external fixation element, a first block fixed to the external fixation element, a second block movably coupled to the first block, and a plurality of pins coupled to the second block.

According to an illustrative embodiment, a bone segment translation system comprises an external fixation element, a pin supporting element movably coupled to the external fixation element, and a plurality of pins connected to the pin supporting element.

According to an illustrative embodiment, a bone segment translation system comprises a frame disposed around a bone of a subject, a first block fixed to the frame, a second block movably coupled to the first block, and one or more pins coupled to the second block.

### BRIEF DESCRIPTION OF THE DRAWINGS

FIG. 1 shows a perspective view of an external fixation device for translation of a bone segment, in accordance with an embodiment.

FIG. 2 shows a right-side view of an external fixation device for translation of a bone segment, in accordance with an embodiment.

FIG. 3 shows a front view of an external fixation device for translation of a bone segment, in accordance with an embodiment.

FIG. 4A shows a perspective view of an external fixation device for translation of a bone segment attached to a bone, in accordance with an embodiment.

FIG. 4B shows a longitudinal axis and a transverse plane with respect to a human body.

FIG. 5A shows an exploded view of a translation portion of an external fixation device for translation of a bone segment, in accordance with an embodiment.

FIG. 5B shows a perspective view of a translation portion of an external fixation device for translation of a bone segment, in accordance with an embodiment.

FIG. 5C shows an exploded view of an upper part of a translation portion of an external fixation device for translation of a bone segment, in accordance with an embodiment.

FIG. 5D shows an exploded view of a lower part of a translation portion of an external fixation device for translation of a bone segment, in accordance with an embodiment.

FIG. 6A shows an exploded view of a motorized translation portion of an external fixation device for translation of a bone segment, in accordance with an embodiment.

FIG. 6B shows a perspective view of a motorized translation portion of an external fixation device for translation of a bone segment, in accordance with an embodiment.

FIG. 6C shows an exploded view of an upper part of a motorized translation portion of an external fixation device for translation of a bone segment, in accordance with an embodiment.

FIG. 7A shows an exploded view of a switch-operated translation portion of an external fixation device for translation of a bone segment, in accordance with an embodiment.

FIG. 7B shows a perspective view of a switch-operated translation portion of an external fixation device for translation of a bone segment, in accordance with an embodiment.

FIG. 7C shows an exploded view of an upper part of a switch-operated translation portion of an external fixation device for translation of a bone segment, in accordance with an embodiment.

FIG. 8 shows a perspective view of an external fixation device for translation of a bone segment coupled with a foot plate, in accordance with an embodiment.

FIG. 9A shows a perspective view of an external fixation device for translation of a bone segment, in accordance with an embodiment.

FIG. 9B shows an enlarged view of a portion of the external fixation device of FIG. 9B, in accordance with an embodiment.

FIG. 10 shows a perspective view of a corticotomy and pin targeting guide, in accordance with an embodiment.

FIG. 11 shows a cross-sectional view of a stroke limiting element, in accordance with an embodiment.

FIGS. 12A and 12B illustrate an external fixation device for translation of a bone segment, in accordance with an alternative embodiment.

FIG. 12C illustrates an enlarged view of a portion of the external fixation device of FIGS. 12A and 12B, in accordance with an embodiment.

FIG. 12D illustrates the external fixation device of FIGS. 12A and 12B around a leg of a patient, in accordance with an embodiment.

FIG. 13A illustrates a perspective view of a corticotomy and pin targeting guide in conjunction with an external fixation device, in accordance with an embodiment.

FIGS. 13B and 13C show perspective views of the corticotomy and pin targeting guide of FIG. 13A, in accordance with an embodiment.

FIGS. 14A and 14B illustrate perspective views of a corticotomy and pin targeting guide in conjunction with an external fixation device, in accordance with an embodiment.

### DETAILED DESCRIPTION

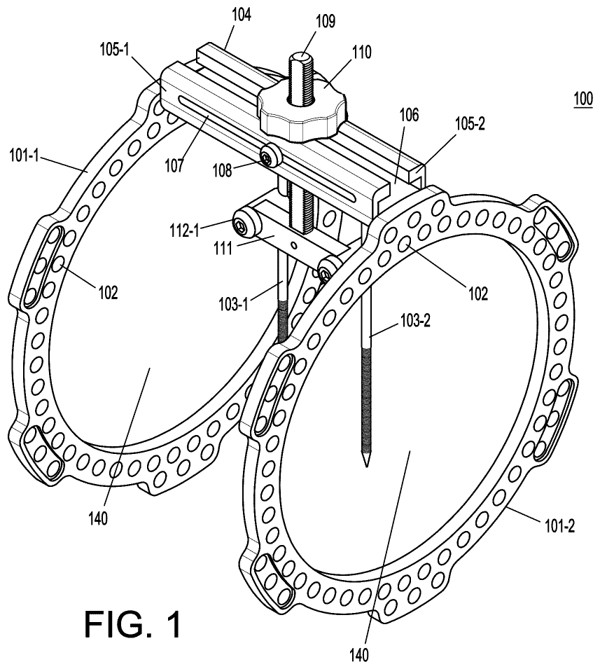

Referring to FIGS. 1-4A, an external fixation device 100 for translation of a bone segment comprises a first external fixation element 101-1 and a second external fixation element 101-2 (collectively, “external fixation elements 101”). In an illustrative embodiment, the external fixation elements 101 comprise circular-shaped frames. However, in other embodiments, the external fixation elements 101 may be, for example, oval-shaped, square-shaped, rectangular-shaped, or any other regular or irregular shape configured for an external fixation application. The external fixation elements 101 respectively comprise a plurality of fastener apertures 102 through which fasteners such as, for example, bolts or screws may be positioned. The external fixation elements 101 respectively further comprise a central aperture 140 that is configured and sized to receive one or more body structures (e.g., a portion of a leg or arm) therethrough.

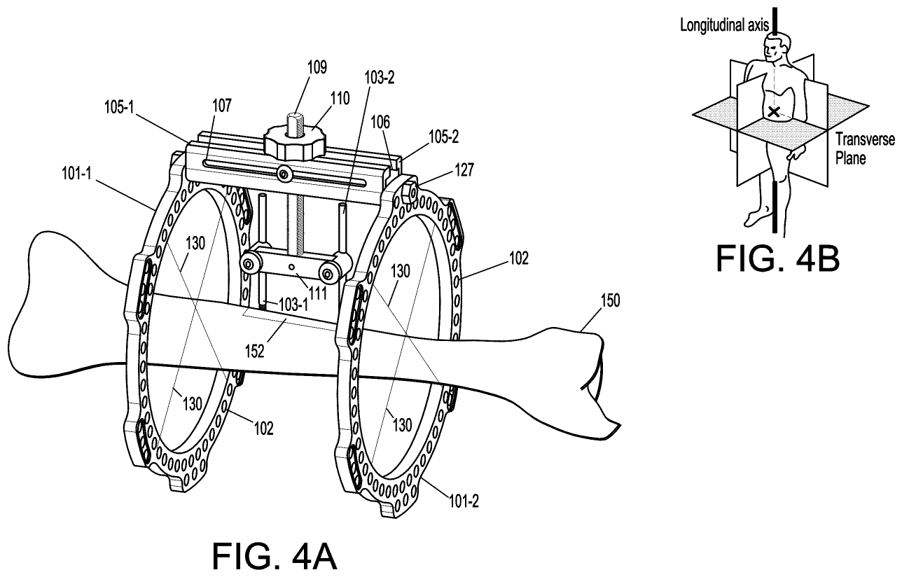

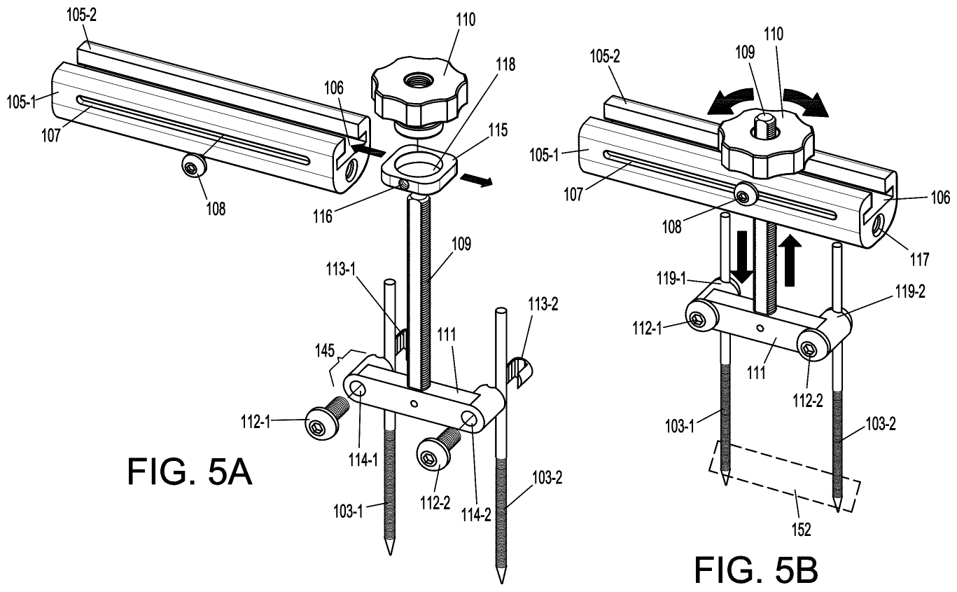

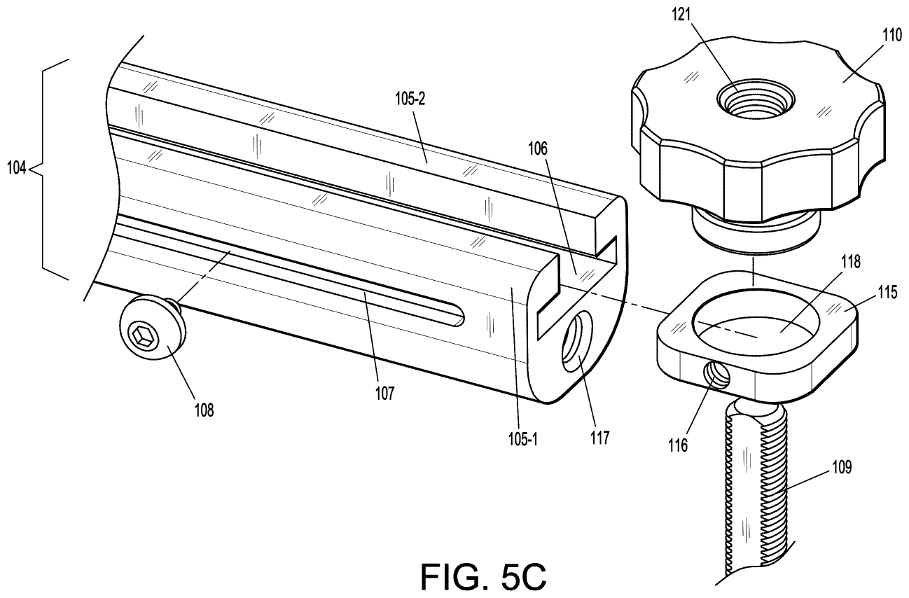

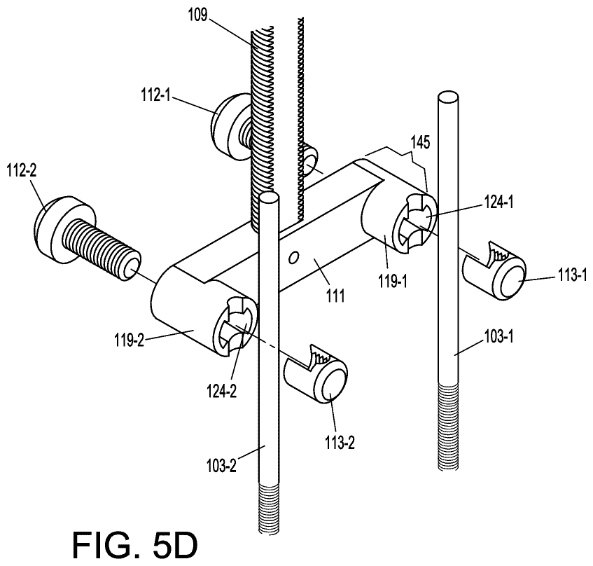

Referring to FIGS. 1-4A and to FIGS. 5A-5D, the external fixation device 100 includes a translation portion including a connecting element 104. The first external fixation element 101-1 and a second external fixation element 101-2 are respectively coupled to right and left sides of the connecting element 104 via respective fasteners inserted through fastener apertures 102 in each of the external fixation elements 101. In more detail, referring to FIGS. 4A, 5B and 5C, a fastener 127 such as, for example, a bolt or screw is inserted through a fastener aperture 102 in the upper portion of second external fixation element 101-2 into a hole 117 in the left side of the connecting element 104 to attach the second external fixation element 101-2 to the connecting element 104. The hole 117 can be threaded to receive a threaded fastener 127, which can be tightened in the hole 117. The first external fixation element 101-1 is similarly attached to the connecting element 104 using a fastener (not shown) similar to the fastener 127 inserted through a fastener aperture 102 in the upper portion of first external fixation element 101-1 into a hole (not shown) in the right side of the connecting element 104.

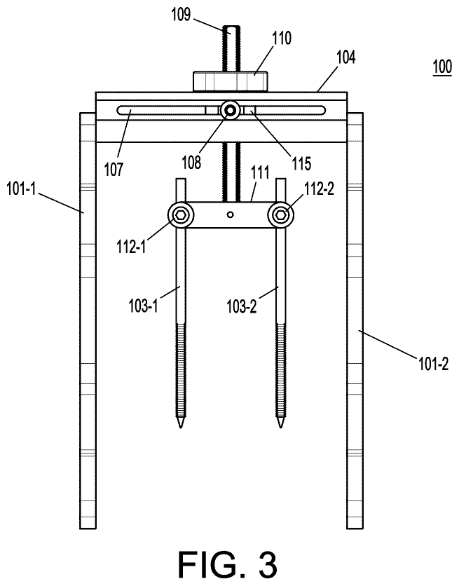

The connecting element 104 includes a front connecting element portion 105-1 and a rear connecting element portion 105-2 which are configured with a top channel portion 106 formed between the front connecting element portion 105-1 and the rear connecting element portion 105-2. The top channel portion 106 has an upside-down T shape. The connecting element 104 further includes a front channel portion 107 formed in the front connecting element portion 105-1. Referring to FIGS. 3, 5A and 5C, the top channel portion 106 is dimensioned to receive a lateral adjustment element 115 therein, which can be slidably inserted into the top channel portion 106 such that the lateral adjustment element 115 can be moved within the top channel portion 106 in the right and left (e.g., lateral) directions as shown by the arrows in FIG. 5A. The lateral adjustment element 115 comprises a central aperture 118 and a hole 116 into which a fastener 108 (e.g., screw or bolt) can be inserted. Similar to the hole 117, the hole 116 can be threaded to receive a threaded fastener 108, which can be tightened in the hole 116. As can be understood from FIGS. 1, 3, 4A and 5A-5C, the fastener 108 is inserted into the front channel portion 107 and into the hole 116. The position of the lateral adjustment element 115 along the top channel portion 106 and the front channel portion 107 can be adjusted prior to tightening the fastener 108 in the hole 116. Once the fastener 108 is tightened in the hole 116, the position of the lateral adjustment element 115 along the top channel portion 106 and the front channel portion 107 is fixed.

The translation portion further includes a transverse adjustment element 110, which is configured and sized to be inserted into the top channel portion 106 and through the central aperture 118 of the lateral adjustment element 115. The transverse adjustment element 110 connects to a translation rod 109, which is attached to and extends from a pin holder 145. The pin holder 145 is configured to support and hold in place a first pin 103-1 and a second pin 103-2 (collectively “pins 103”) mounted to the pin holder 145. Although two pins 103 are shown, in different embodiments, the pin holder 145 may be configured to include one pin 103 or more than two pins 103 mounted thereto.

The connecting element 104 includes an opening (not shown) in the underside of the connecting element 104 extending in the left and right directions through which the translation rod 109 can be inserted through the central aperture 118 to meet with and connect to the transverse adjustment element 110. As can be understood from FIG. 5C, the transverse adjustment element 110 comprises a threaded knob, and the translation rod 109 is threaded so that the transverse adjustment element 110 can be screwed on to the translation rod 109 with mating threads 121 formed in an open central portion of the transverse adjustment element 110. As shown by a first set of arrows in FIG. 5B, turning (rotating) the transverse adjustment element 110 in the clockwise direction while mated with the translation rod 109 causes the translation rod 109 to move in a first transverse direction (e.g., along the transverse plane) away from a bone 150 (see FIGS. 4A and 4B). Alternatively, as shown by a second set of arrows in FIG. 5B turning (rotating) the transverse adjustment element 110 in the counter-clockwise direction while mated with the translation rod 109 causes the translation rod 109 to move in a second (opposite) transverse direction (e.g., along the transverse plane in an opposite direction) toward the bone 150. The translation rod 109 moves in a direction perpendicular to the longitudinal axis shown in FIG. 4B, which is also perpendicular to upper and lower surfaces of the transverse adjustment element 110. Since the translation rod 109 is attached to and extends from the pin holder 145, the pin holder 145 moves in the same direction as the translation rod 109 (e.g., away from or toward the bone 150 along the transverse axis perpendicular to the bone 150).

As used herein, the term “perpendicular” may refer to at an angle of 90 degrees with respect to the longitudinal axis or with respect to a lengthwise direction of bone or within a tolerance range of, for example, 90±5 degrees or more depending on anatomical conditions of a subject.

As used herein, the terms “transverse” or “transverse plane” may refer to at an angle of 90 degrees with respect to the longitudinal axis or with respect to a lengthwise direction of bone or within a tolerance range of, for example, ±5 degrees or more depending on anatomical conditions of a subject.

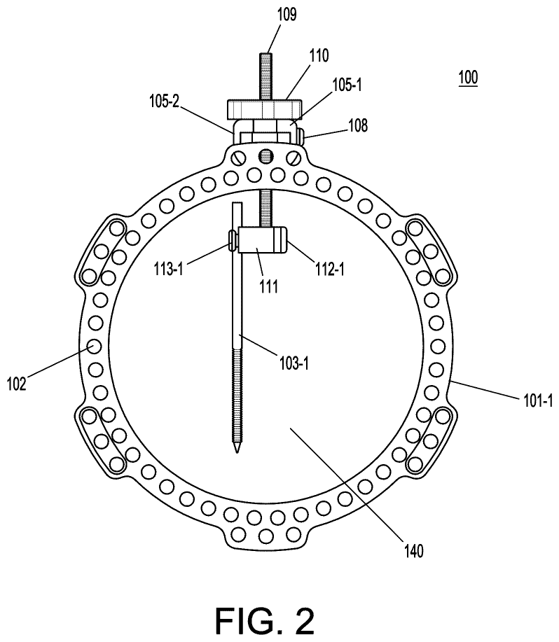

Referring to FIGS. 1-3, 4A, 5A, 5B and 5D, the pin holder 145 comprises a body portion 111 comprising a first hole 114-1 and a second hole 114-2 adjacent right and left edges of the body portion 111. The first and second holes 114-1 and 114-2 are formed in a first extension portion 119-1 and a second extension portion 119-2, respectively. The first and second extension portions 119-1 and 119-2 respectively include first and second openings 124-1 and 124-2 at back ends thereof. The first and second holes 114-1 and 114-2 are configured to receive a first fastener 112-1 and a second fastener 112-2, respectively. Similar to the other fasteners described herein, the first and second fasteners 112-1 and 112-2 can be, for example, screws or bolts. In an illustrative embodiment, at least portions of the first and second holes 114-1 and 114-2 are threaded so that the first and second fasteners 112-1 and 112-2 can be screwed into and secured in the first and second holes 114-1 and 114-2. The first and second fasteners 112-1 and 112-2 are configured to mate with a first pin support 113-1 and a second pin support 113-2, respectively. The first and second pin supports 113-1 and 113-2 are configured to fit around the first and second pins 103-1 and 103-2, respectively, and fit securely in the first and second openings 124-1 and 124-2 at the back ends of respective first and second extension portions 119-1 and 119-2. In an illustrative embodiment, at least portions of the interior surfaces of the first and second pin supports 113-1 and 113-2 are threaded so that the leading edges of the first and second fasteners 112-1 and 112-2 can mate with first and second pin supports 113-1 and 113-2 and secure the first and second pin supports 113-1 and 113-2 in the first and second openings 124-1 and 124-2. As shown, for example, in FIGS. 2-4A and 5B, the securing of the first and second pin supports 113-1 and 113-2 in the first and second openings 124-1 and 124-2 fixes the first and second pins 103-1 and 103-2 in a desired position to the body portion 111 of the pin holder 145.

The bone segment translation system comprises the external fixation device 100 including the first external fixation element 101-1 and a second external fixation element 101-2. In illustrative embodiments, an affected limb of a patient (e.g., human subject) is received through the central apertures 140 of the first and second external fixation elements 101-1 and 101-2. As shown in FIG. 4A, one or more wires 130 may extend across the first and second external fixation elements 101-1 and 101-2 through the patient's affected limb (e.g., through the bone 150 and/or soft tissue). The wires 130 may be connected to the first and second external fixation elements 101-1 and 101-2 with fasteners such as, for example, nuts and bolts.

Referring to FIG. 4A, in illustrative embodiments, the first and second pins 103-1 and 103-2 are positioned through soft tissue of a patient into a bone segment 152. In the case of, for example, a tibia, the bone segment 152 is cut from the topography of the metaphysis region of the bone 150 where the bone 150 is highly vascularized and engorged with blood and nutrients capable of delivering healing factors from the proximal tibia to the distal tibia. As a result, the nutrients and cells that flow to the lower limb (e.g., foot and ankle) are concentrated, thereby enhancing bone remodeling, arthrodesis, and soft tissue healing. In illustrative embodiments, translation of the bone segment 152 effectively creates an osseus compression pump, which actively pumps and promotes the flow of blood and nutrients through the bone 150 to an extremity region. Similar techniques can apply to bones other than the tibia (e.g., other long bones) where the blood supply is present. Flow can be in the proximal to distal direction or reversed, depending on the location of the bone segment 152.

In accordance with illustrative embodiments, the bone segment translation system comprising the external fixation device 100 moves (translates) the bone segment 152 transversely with respect to the bone 150 (e.g., perpendicular to the bone 150 (e.g., along the transverse axis shown in FIG. 4B). The bone segment 152 is transversely distracted a designated distance away from the bone 150. In a non-limiting illustrative example, the bone segment 152 can be distracted in the range of greater than 0 mm to 10 mm away from its original position, and then after some time depending on clinical considerations and/or patient response (e.g., a specified number of hours, days, weeks) moved back (e.g., transversely retracted) a designated distance back toward the original anatomical position. The designated retraction distance can be less than or equal to the original distraction distance. In addition, the transverse distraction and/or traction (retraction) can be accomplished in multiple steps. For example, the transverse distraction and/or retraction of the bone segment 152 can be performed incrementally. In more detail, the bone segment 152 can be transversely distracted in stages (e.g., moved a first distance away from an original anatomical position, then after some time elapses, moved to a second distance farther away from the original anatomical position, etc.) until the bone segment 152 reaches a designated distance away from the original anatomical position. Similarly, the bone segment 152 can be transversely retracted in stages (e.g., moved a first distance toward the original anatomical position, then after some time elapses, moved to a second distance closer to the original position, etc.) until the bone segment 152 reaches a designated distance closer to the original anatomical position or returns to the original anatomical position. The range of greater than 0 mm to 10 mm is merely an example and may vary depending on the needs and/or progress of a given patient.

The translation portion described herein above is used to translate (e.g., distract and retract) the bone segment 152 to the desired positions along the transverse axis. In further detail, once the first and second pins 103-1 and 103-2 are lodged or otherwise affixed to the bone segment 152, the translation rod 109 in combination with the transverse adjustment element 110 functions in a manner similar to a cammed shaft to translate the bone segment 152 to the desired positions along the transverse axis. As explained herein in connection with FIGS. 5B and 5C, the translation rod 109 is threaded so that the transverse adjustment element 110 can be screwed on to the translation rod 109. As shown by the first set of arrows in FIG. 5B, turning the transverse adjustment element 110 in the clockwise direction while mated with the translation rod 109 causes the translation rod 109 to be distracted in a first transverse direction away from a bone 150. As a result, the attached pin holder 145, corresponding first and second pins 103-1 and 103-2 and the bone segment 152 move in the first transverse direction consistent with the movement of the translation rod 109. As shown by the second set of arrows in FIG. 5B, turning the transverse adjustment element 110 in the counter-clockwise direction while mated with the translation rod 109 causes the translation rod 109, attached pin holder 145, corresponding first and second pins 103-1 and 103-2 and the bone segment 152 to move (be retracted) in a second opposite transverse direction toward the bone 150.

In illustrative embodiments, the transverse adjustment element 110 may include an indexing mechanism to precisely control the stroke per turn. The transverse adjustment element 110 may include a haptic type of feedback to notify a user of when to stop rotation. The translation rod 109 may be equipped with a clutching mechanism to prevent overloading or fracturing of the bone segment 152. In some illustrative embodiments, the translation rod 109 and/or transverse adjustment element 110 may be controlled by a programmable stopping mechanism that prevents overtravel that could potentially break through the skin and controls the stroke of the transverse adjustment element 110 as it returns the bone segment 152 to its original anatomical position. The programmable stopping mechanism may be electronically linked to the translation rod 109 and/or transverse adjustment element 110. In some embodiments, the programmable stopping mechanism may be remotely controlled such as through an application on a computer or mobile device.

The shape of the bone segment 152 can vary. For example, the shape of the bone segment 152 can be rectangular, round or any conceivable profile or pattern. The resulting effect of the transverse distraction of a bone segment is the enhanced supply of blood delivered through the endosteal cavity via the medullary canal, thereby delivering nutrients through arteries and marrow sinusoids before exiting via numerous small vessels that branch through a cortex. The bone from which a bone segment 152 can be translated may be any one of multiple bones in a human body such as, for example, the tibia, femur, humerus, radius and ulna. Bones, such as, for example, long bones, receive blood supply from multiple sources, including the central nutrient artery, the metaphyseal-epiphyseal arteries, which enter long bones near their distal ends, and the periosteal arteries. In the illustrative embodiments, distraction of the bone segment 152 is used to thrust the flow of blood and nutrients through the bone 150 to an extremity region.

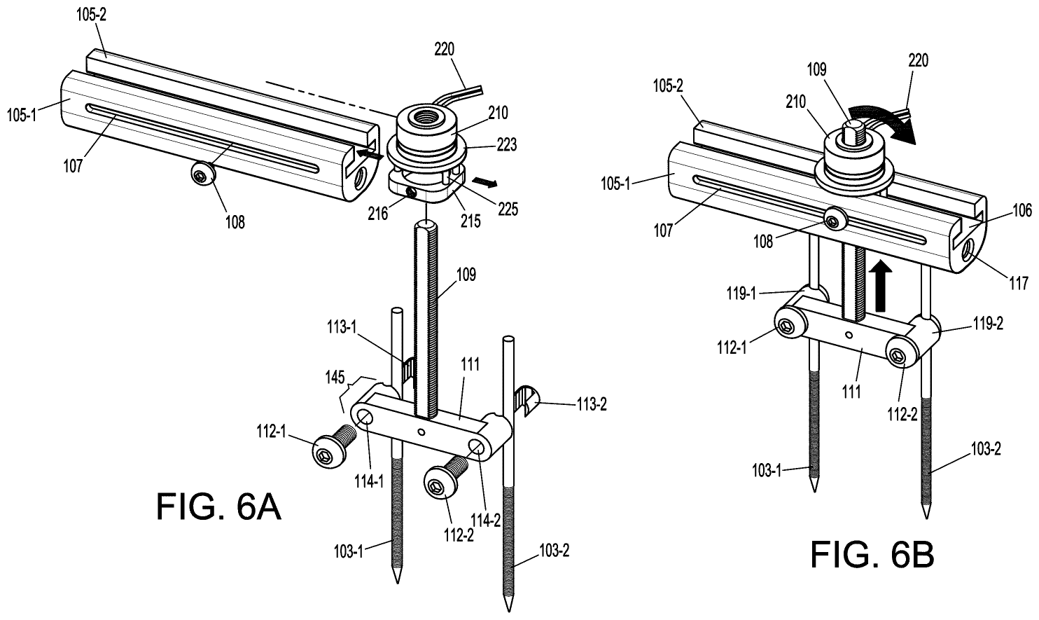

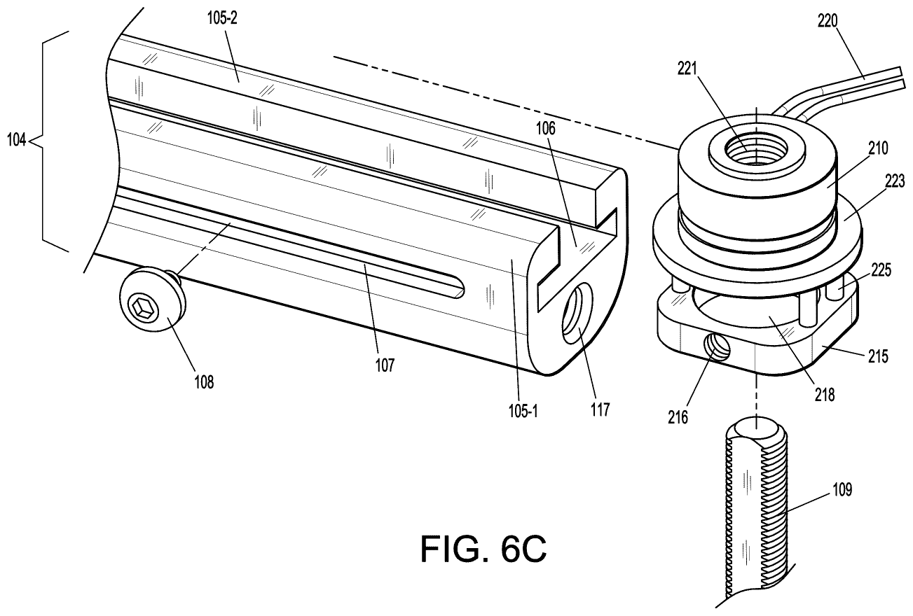

FIGS. 6A-6C illustrate an alternative embodiment of the translation portion, where the component for causing movement of the translation rod 109 comprises motorized transverse adjustment element 210 instead of the transverse adjustment element 110, which is manually turned. Components comprising the same reference numbers in FIGS. 6A-6C as those used in FIGS. 1-5D are the same as the components previously described in connection with FIGS. 1-5D. As can be seen in FIGS. 6A-6C, the motorized transverse adjustment element 210 includes a motor, which is electrically connected to a voltage source via one or more wires 220. The motor is configured to turn the motorized transverse adjustment element 210 in the clockwise or counter-clockwise direction as a substitute for manual turning. The motorized transverse adjustment element 210 includes a platform portion 223, which is connected to a lateral adjustment element 215 via column portions 225 disposed between and connected to both the platform portion 223 and the lateral adjustment element 215. Similar to the lateral adjustment element 115, the top channel portion 106 is dimensioned to receive the lateral adjustment element 215 therein, which can be slidably inserted into the top channel portion 106 such that the lateral adjustment element 215 can be moved within the top channel portion 106 in the right and left (e.g., lateral) directions as shown by the arrows in FIG. 6A. The platform portion 223 rests on and slides along upper surfaces of the front connecting element portion 105-1 and a rear connecting element portion 105-2. The lateral adjustment element 215 comprises a central aperture 218 and a hole 216 into which a fastener 108 (e.g., screw or bolt) can be inserted. Similar to the hole 117, the hole 216 can be threaded to receive a threaded fastener 108, which can be tightened in the hole 216. As can be understood from FIGS. 6A-6C, the fastener 108 is inserted into the front channel portion 107 and into the hole 216. The position of the lateral adjustment element 215 along the top channel portion 106 and the front channel portion 107 can be adjusted prior to tightening the fastener 108 in the hole 216. Once the fastener 108 is tightened in the hole 216, the position of the lateral adjustment element 215 along the top channel portion 106 and the front channel portion 107 is fixed.

The motorized transverse adjustment element 210 connects to the translation rod 109, which is attached to and extends from the pin holder 145. The connecting element 104 includes an opening (not shown) in the underside of the connecting element 104 extending in the left and right directions through which the translation rod 109 can be inserted to extend through the central aperture 218 to meet with and connect to the motorized transverse adjustment element 210. As can be understood from FIG. 6C, the translation rod 109 is threaded so that the motorized transverse adjustment element 210 can be screwed on to the translation rod 109 with mating threads 221 formed in the motorized transverse adjustment element 210. As shown by the arrows in FIG. 6B, motorized turning (rotating) of the motorized transverse adjustment element 210 in the clockwise direction while mated with the translation rod 109 causes the translation rod 109 to move in a first transverse direction along the transverse axis (e.g., to be distracted) away from and perpendicular to a bone 150. Alternatively, motorized turning (rotating) of the motorized transverse adjustment element 210 in the counter-clockwise direction while mated with the translation rod 109 causes the translation rod 109 to move a second opposite transverse direction along the transverse axis (e.g., to be retracted) toward and perpendicular to the bone 150. As can be understood, the motor has at least forward (e.g., clockwise) and backward (e.g., counter-clockwise) speeds. The translation rod 109 moves in a direction along the transverse axis perpendicular to upper and lower surfaces of the motorized transverse adjustment element 210. Since the translation rod 109 is attached to and extends from the pin holder 145, the pin holder 145 moves in the same direction as the translation rod 109 (e.g., away from or toward the bone 150 in a transverse direction with respect to the bone 150).

Similar to the operation with the transverse adjustment element 110, once the first and second pins 103-1 and 103-2 are lodged or otherwise affixed to the bone segment 152, the translation rod 109 in combination with the motorized transverse adjustment element 210 functions in a manner similar to a cammed shaft to distract and return (e.g., retract) the bone segment 152 to the desired positions. As explained herein in connection with FIGS. 6B and 6C, the translation rod 109 is threaded so that the motorized transverse adjustment element 210 can be screwed on to the translation rod 109. As shown by the arrows in FIG. 6B, motorized turning of the motorized transverse adjustment element 210 in the clockwise direction while mated with the translation rod 109 causes the translation rod 109 to be distracted in a first transverse direction away from a bone 150. As a result, the attached pin holder 145, corresponding first and second pins 103-1 and 103-2 and the bone segment 152 move in the transverse direction consistent with the movement of the translation rod 109. Motorized turning of the motorized transverse adjustment element 210 in the counter-clockwise direction while mated with the translation rod 109 causes the translation rod 109, attached pin holder 145, corresponding first and second pins 103-1 and 103-2 and the bone segment 152 to move in the second opposite transverse direction (e.g., to be retracted) with respect to the bone 150.

In illustrative embodiments, the motorized transverse adjustment element 210 may include an indexing mechanism to precisely control the stroke per turn. Like when used with the transverse adjustment element 110, when used with the motorized transverse adjustment element 210, the translation rod 109 may be equipped with a clutching mechanism to prevent overloading or fracturing of the bone segment 152. In some illustrative embodiments, the translation rod 109 and/or motorized transverse adjustment element 210 may be controlled by a programmable stopping mechanism that prevents overtravel that could potentially break through the skin and controls the stroke of the motorized transverse adjustment element 210 as it returns the bone segment 152 to its original anatomical position. The programmable stopping mechanism may be electronically linked to the translation rod 109 and/or motorized transverse adjustment element 210. In some embodiments, the programmable stopping mechanism may be remotely controlled such as through an application on a computer or mobile device.

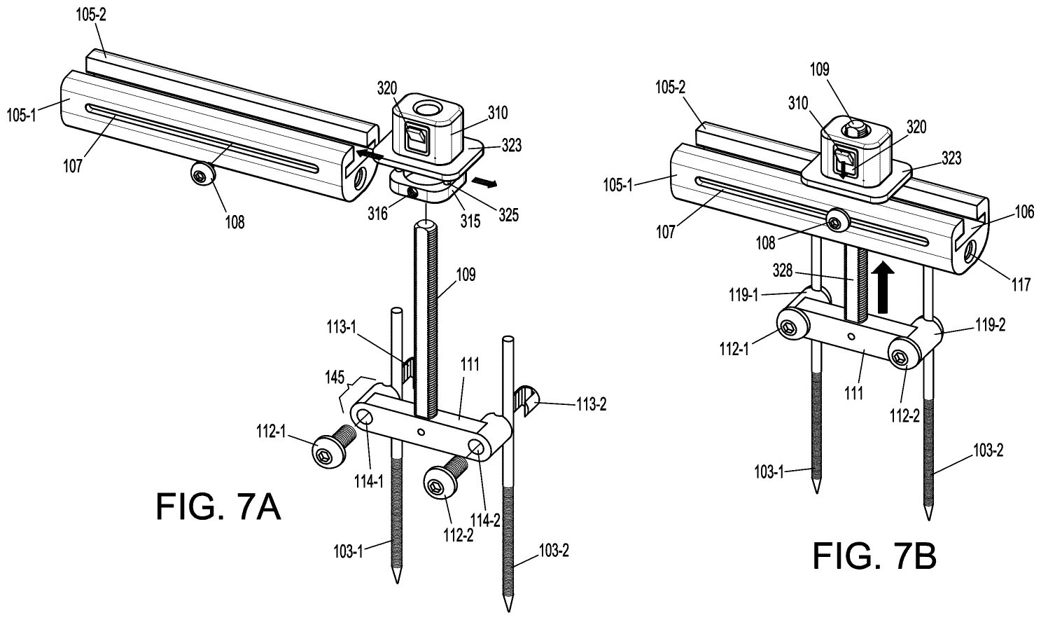

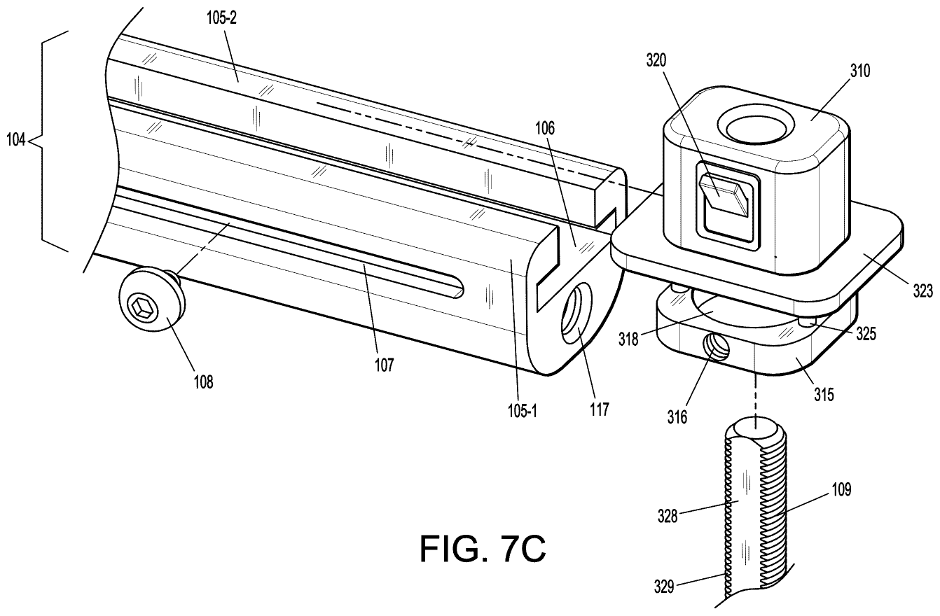

FIGS. 7A-7C illustrate another alternative embodiment of the translation portion, where the component for causing movement of the translation rod 109 comprises switch-operated transverse adjustment element 310 instead of the transverse adjustment element 110 or the motorized transverse adjustment element 210. Components comprising the same reference numbers in FIGS. 7A-7C as those used in FIGS. 1-5D or FIGS. 6A-6C are the same as the components previously described in connection with FIGS. 1-5D or FIGS. 6A-6C. As can be seen in FIGS. 7A-7C, the switch-operated transverse adjustment element 310 (also referred to herein as a “switch assembly”) includes a switch element 320. The switch element 320 is configured to function as a ratchet or winch-like mechanism that when moved in the up and down directions either ratchets the translation rod 109 in the transverse direction away from the bone 150 or in the opposite transverse direction toward the bone 150. The switch element 320 includes an internal mechanism controlled by an outer selector switch (not shown) to control whether operation of the switch element 320 in the up and down directions causes the translation rod 109 to move in the transverse direction away from the bone 150 or in the opposite transverse direction toward the bone 150. The switch-operated transverse adjustment element 310 includes a platform portion 323, which is connected to a lateral adjustment element 315 via column portions 325 disposed between and connected to both the platform portion 323 and the lateral adjustment element 315. Similar to the lateral adjustment element 215, the top channel portion 106 is dimensioned to receive the lateral adjustment element 315 therein, which can be slidably inserted into the top channel portion 106 such that the lateral adjustment element 315 can be moved within the top channel portion 106 in the right and left (e.g., lateral) directions as shown by the arrows in FIG. 7A. The platform portion 323 rests on and slides along upper surfaces of the front connecting element portion 105-1 and a rear connecting element portion 105-2. The lateral adjustment element 315 comprises a central aperture 318 and a hole 316 into which a fastener 108 (e.g., screw or bolt) can be inserted. Similar to the hole 117, the hole 316 can be threaded to receive a threaded fastener 108, which can be tightened in the hole 316. As can be understood from FIGS. 7A-7C, the fastener 108 is inserted into the front channel portion 107 and into the hole 316. The position of the lateral adjustment element 315 along the top channel portion 106 and the front channel portion 107 can be adjusted prior to tightening the fastener 108 in the hole 316. Once the fastener 108 is tightened in the hole 316, the position of the lateral adjustment element 315 along the top channel portion 106 and the front channel portion 107 is fixed.

The switch-operated transverse adjustment element 310 connects to the translation rod 109, which is attached to and extends from the pin holder 145. The connecting element 104 includes an opening (not shown) in the underside of the connecting element 104 extending in the left and right directions through which the translation rod 109 can be inserted to extend through the central aperture 318 to meet with and connect to the switch-operated transverse adjustment element 310. As can be understood from FIGS. 7A-7C, the translation rod 109 includes a grooved portion 328 where the threads end and form tooth portions 329. The tooth portions 329 engage with internal complementary tooth portions of the switch-operated transverse adjustment element 310 that are coupled to the switch element 320. When the switch element 320 is operated, the internal complementary tooth portions move the translation rod in a transverse direction away from the bone 150 or in an opposite transverse direction toward the bone 150 by virtue of the engagement of the tooth portions 329 with the internal complementary tooth portions of the switch-operated transverse adjustment element 310. As shown by the down and up arrows in FIG. 7B, toggling the switch in the downward direction causes the translation rod 109 to be distracted in a transverse direction away from the bone 150. The translation rod 109 moves in the transverse directions perpendicular to the bone 150, which is also perpendicular to upper and lower surfaces of the switch-operated transverse adjustment element 310. Since the translation rod 109 is attached to and extends from the pin holder 145, the pin holder 145 moves in the same direction as the translation rod 109 (e.g., away from or toward the bone 150 in a transverse direction with respect to the bone 150).

Once the first and second pins 103-1 and 103-2 are lodged or otherwise affixed to the bone segment 152, the translation rod 109 in combination with the switch-operated transverse adjustment element 310 functions in a manner similar to a ratchet or winch to distract and return (retract) the bone segment 152 to the desired positions. As explained herein in connection with FIGS. 7B and 7C, the translation rod 109 includes a grooved portion 328 where the threads end and form tooth portions 329. The tooth portions 329 engage with internal complementary tooth portions of the switch-operated transverse adjustment element 310 that are coupled to the switch element 320. When the switch element 320 is operated, the internal complementary tooth portions move the translation rod 109 in transverse directions by virtue of the engagement of the tooth portions 329 with the internal complementary tooth portions of the switch-operated transverse adjustment element 310. As a result, the attached pin holder 145, corresponding first and second pins 103-1 and 103-2 and the bone segment 152 move in a direction consistent with the movement of the translation rod 109. Manual toggling of the switch element 320 while the switch-operated transverse adjustment element 310 is mated with the tooth portions 329 of the translation rod 109 causes the translation rod 109, attached pin holder 145, corresponding first and second pins 103-1 and 103-2 and the bone segment 152 to move in a first transverse direction or second (opposite the first) transverse direction with respect to the bone 150.

In illustrative embodiments, the switch-operated transverse adjustment element 310 may include an indexing mechanism to precisely control the stroke per turn. Like when used with the transverse adjustment element 110, when used with the switch-operated transverse adjustment element 310, the translation rod 109 may be equipped with a clutching mechanism to prevent overloading or fracturing of the bone segment 152. In some illustrative embodiments, the translation rod 109 and/or switch-operated transverse adjustment element 310 may be controlled by a programmable stopping mechanism that prevents overtravel that could potentially break through the skin and controls the stroke of the switch-operated transverse adjustment element 310 as it returns the bone segment 152 to its original anatomical position. The programmable stopping mechanism may be electronically linked to the translation rod 109 and/or switch-operated transverse adjustment element 310. In some embodiments, the programmable stopping mechanism may be remotely controlled such as through an application on a computer or mobile device.

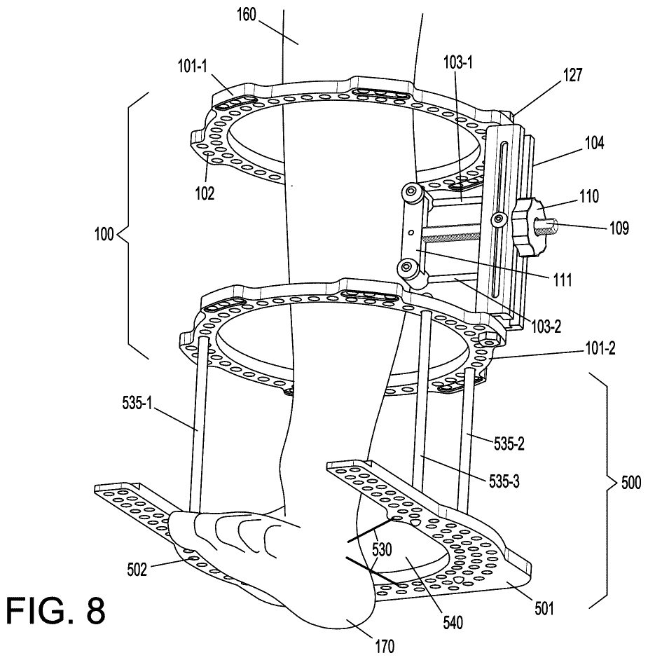

Referring to FIG. 8, the external fixation device 100 is affixed to a leg 160 of a human subject and is coupled to a footplate device 500, which functions to stabilize a foot 170 of the human subject in a particular position. The external fixation device 100 is coupled to the footplate device 500 via one or more connection struts 535-1, 535-2 and 535-3 (collectively “connection struts 535”) extending between the second external fixation element 101-2 and a footplate 501. In an illustrative embodiment, the footplate 501 comprises a U-shaped frame. However, in other embodiments, the footplate 501 may be, for example, oval-shaped, square-shaped, rectangular-shaped, ring-shaped or any other regular or irregular shape configured for an external fixation application. Like the external fixation elements 101, the footplate 501 comprises a plurality of fastener apertures 502 through which fasteners such as, for example, bolts or screws may be positioned to secure the connection struts 535. The connection struts 535 themselves may be positioned through the fastener apertures 102 and 502 to be secured to the footplate 501 and to the second external fixation element 101-2. The footplate 501 further comprises a central aperture 540 that is configured and sized to receive one or more body structures (e.g., a portion of a leg 160 or foot 170) therethrough.

As shown in FIG. 8, one or more wires 530 may extend from the footplate 501 through the patient's affected leg 160, ankle and/or foot 170 (e.g., through the bone 150, other bones and/or soft tissue). The wires 530 may be connected to the footplate 501 with fasteners such as, for example, nuts and bolts. The wires 530 may be used in different orientations and angles to stabilize the foot 170 in a particular position or to move the foot 170 into a desired position over time. Although two wires 530 and three connection struts 535 are shown, the embodiments are not necessarily limited thereto, and more or less wires 530 and connection struts 535 may be used.

In some illustrative embodiments, in connection with the external fixation device 100, the connecting element 104 may be connected to one of the first external fixation element 101-1 and the second external fixation element 101-2, and the remaining external fixation element can be omitted.

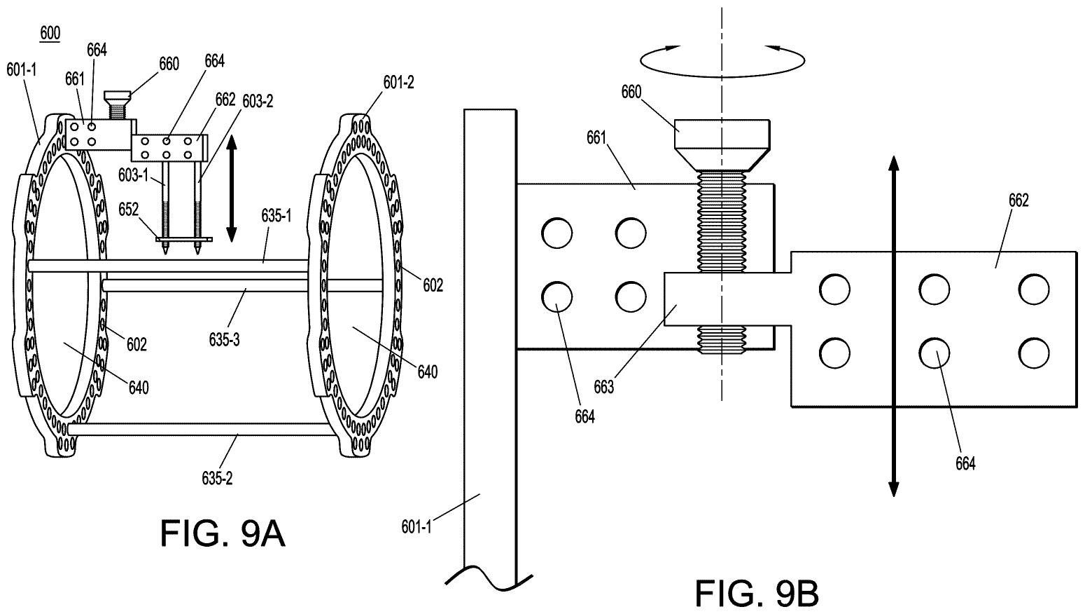

FIG. 9A illustrates an external fixation device 600 for translation of a bone segment 652, in accordance with an alternative embodiment. FIG. 9B illustrates an enlarged portion of FIG. 9A. Elements the same or similar to those described in connection with previous embodiments are designated by similar reference numbers. Like the external fixation device 100, the external fixation device 600 includes a first external fixation element 601-1 and a second external fixation element 601-2 (collectively, “external fixation elements 601”). In an illustrative embodiment, the external fixation elements 601 comprise circular-shaped frames. However, in other embodiments, the external fixation elements 601 may be, for example, oval-shaped, square-shaped, rectangular-shaped, or any other regular or irregular shape configured for an external fixation application. The external fixation elements 601 respectively comprise a plurality of fastener apertures 602 through which fasteners such as, for example, bolts or screws may be positioned. The external fixation elements 601 respectively further comprise a central aperture 640 that is configured and sized to receive one or more body structures (e.g., a portion of a leg or arm) therethrough.

The first external fixation element 601-1 and the second external fixation element 601-2 are coupled to each other via one or more connection struts 635-1, 635-2 and 635-3 (collectively “connection struts 635”) extending between the first and second external fixation elements 601-1 and 602-2. Fasteners such as, for example, bolts or screws may be positioned through the fastener apertures 602 to secure the connection struts 635. The connection struts 635 themselves may be positioned through the fastener apertures 602 to be secured to the first and second external fixation elements 601-1 and 601-2. Although three connection struts 635 are shown, the embodiments are not necessarily limited thereto, and more or less connection struts 635 may be used.

A fixed block 661 is secured to the first external fixation element 601-1 via one or more fasteners inserted through respective fastener apertures 602 in the first external fixation element 601-1 and corresponding respective fastener apertures 664 in the fixed block 661. The fixed block 661 is coupled to a translating block 662 via a protruding portion 663 extending from the translating block 662. The protruding portion 663 includes a threaded hole (not shown) in the protruding portion 663 to mate with a worm screw 660 disposed through a threaded fastener aperture 664 in the fixed block. Similar to the transverse adjustment element 110, rotation of the worm screw 660, by virtue of its engagement with the protruding portion 663 causes the translating block 662 to move in first and second transverse directions (e.g., along the transverse plane) perpendicular to the longitudinal axis and to a longitudinal direction of a bone. For example, referring to the arrows in FIGS. 9A and 9B, turning (rotating) the worm screw 660 in the clockwise direction while mated with the protruding portion 663 causes the translating block 662 to move in a first transverse direction (e.g., along the transverse plane) away from a bone. Alternatively, referring to the arrows in FIGS. 9A and 9B, turning (rotating) the worm screw 660 in the counter-clockwise direction while mated with the protruding portion 663 causes the translating block 662 to move in a second (opposite) transverse direction (e.g., along the transverse plane in an opposite direction) toward the bone. The translating block 662 moves in a direction perpendicular to the longitudinal axis shown in FIG. 4B.

As shown in FIG. 9A, first and second pins 603-1 and 603-2 are attached to the translating block 662. The first and second pins 603-1 and 603-2 are attached to the translating block 662 through respective fastener apertures 664 formed in the translating block 662. For example, the respective fastener apertures 664 and first and second pins 603-1 and 603-2 can be threaded so that the first and second pins 603-1 and 603-2 can be screwed into the respective fastener apertures 664 to secure the first and second pins 603-1 and 603-2 to the translating block 662. Similar to the first and second pins 103-1 and 103-2, the first and second pins 603-1 and 603-2 engage and are secured to a bone segment 652, which is like the bone segment 152.

As a result, when the translating block 662 is translated, the attached corresponding first and second pins 603-1 and 603-2 and the bone segment 652 move in a direction consistent with the movement of the translating block 662. For example, turning (rotating) the worm screw 660 in the clockwise direction while mated with the protruding portion 663 causes the translating block 662, attached first and second pins 603-1 and 603-2 and the bone segment 652 to move in a first transverse direction (e.g., along the transverse plane) away from a bone. Alternatively, turning (rotating) the worm screw 660 in the counter-clockwise direction while mated with the protruding portion 663 causes the translating block 662, attached first and second pins 603-1 and 603-2 and the bone segment 652 to move in a second (opposite) transverse direction (e.g., along the transverse plane in an opposite direction) toward the bone.

In illustrative embodiments, the worm screw 660 may include an indexing mechanism to precisely control the stroke per turn. The worm screw 660 may include a haptic type of feedback to notify a user of when to stop rotation. The worm screw 660 may be equipped with a clutching mechanism to prevent overloading or fracturing of the bone segment 652. In some illustrative embodiments, the worm screw 660 may be controlled by a programmable stopping mechanism that prevents overtravel that could potentially break through the skin and controls the stroke of the worm screw 660 as it returns the bone segment 652 to its original anatomical position. The programmable stopping mechanism may be electronically linked to the worm screw 660. In some embodiments, the programmable stopping mechanism may be remotely controlled such as through an application on a computer or mobile device. The external fixation device 600 is not limited to a worm screw 660 and may include other mechanisms to effect rotation and corresponding movement of the translating block 662 such as, for example, a motor or switch-operated mechanism similar to the motorized and switch-operated transverse adjustment elements 210 and 310. In some illustrative embodiments, in connection with the external fixation device 600, the second external fixation element 601-2 can be omitted.

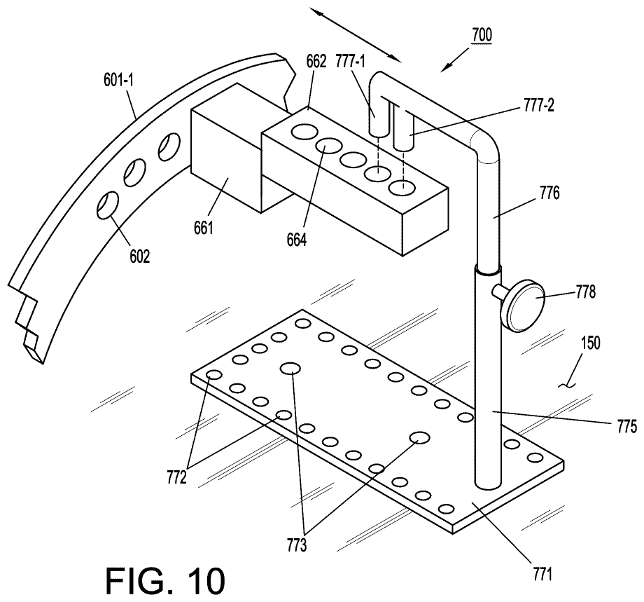

Referring to FIG. 10, a corticotomy and pin targeting guide 700, when fitted to the translating block 662 by way of a first peg 777-1 and second peg 777-2 through corresponding fastener apertures 664 in the translating block 662 assures alignment of first and second pins 603-1 and 603-2 with a corticotomy in a bone (e.g., bone 150). The corticotomy and pin targeting guide 700 includes a plurality of the drill holes 772 through a base plate 771 around the perimeter of the base plate 771.

The corticotomy and pin targeting guide 700 aligns the translating block 662 such that the first and second pins 603-1 and 603-2 can be perfectly placed or substantially perfectly placed in the center of the corticotomy. The drill holes 772 allow a surgeon to perforate the bone 150 in a perfect or substantially perfect rectangle of a known dimension to create the cortical window. Multiple sizes and drill hole configurations for the base plate 771 are contemplated to assure that drill holes 772 permit a surgeon to create a cortical window that is directly underneath the translating block 662 to which the first and second pins 603-1 and 603-2 are to be affixed. In this way, the first and second pins 603-1 and 603-2 can be accurately placed and fixed to the cortical window.

The corticotomy and pin targeting guide 700 includes a base rod 775 extending perpendicularly from the base plate 771, and an L-shaped registration rod 776, which fits into the base rod 775. The height of the L-shaped registration rod 776 is adjustable in a telescoping manner and can be fixed at a given height over the bone 150 and base plate 771 by a threaded knob 778, which can be hand tightened. The first and second pegs 777-1 and 777-2 extending from the L-shaped registration rod 776 can be aligned with different sets of fastener apertures 664 in the translating block 662. Once the first and second pins 603-1 and 603-2 positioned in the bone and placed on the translating block 662, the cortical window is created and the corticotomy and pin targeting guide 700 is removed.

The corticotomy and pin targeting guide 700 further includes a plurality of Kirschner wire (K-wire) holes 773. The K-wire holes can be used to align with points on a bone 150 where K-wires need to be inserted into the bone 150 for stabilization of the corticotomy and pin targeting guide 700 and bone 150 during creation of the cortical window. K-wires can be inserted through the K-wire holes 773 into the bone 150 and removed following creation of the cortical window.

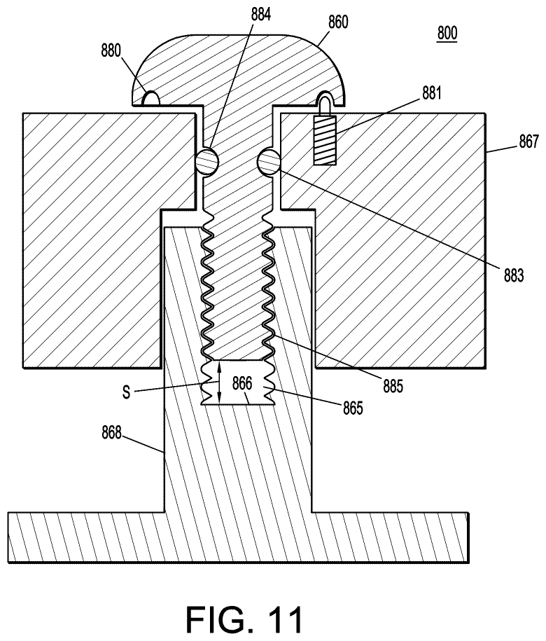

Referring to FIG. 11, a stroke limiting element 800 limits the stroke distance S (distance of travel) that a translating block 662 translates. A screw 860 that actuates the translation, which is the same or similar to the worm screw 660 described in connection with FIGS. 9A and 9B, is rotated to create the translation until such a point whereby the screw 860 bottoms out after traveling the designated stroke distance S, thus stopping any further translation. The stroke distance S can be, for example, surgeon defined to avoid over translation.

The screw 860 is received in a receiving portion 868, which includes a threaded aperture 865. The screw 860 includes threads 885 to mate with the threads 885 in the threaded aperture 865. In an illustrative embodiment, the receiving portion 868 is part of the translating block 662. In another illustrative embodiment, the receiving portion 868 is part of the fixed block 661. The screw 860 has a defined distance of travel (stroke distance S) as set by the depth of the threaded aperture 865. For example, when the screw reaches a floor 866 of the threaded aperture 865, further rotation of the screw 860 is prevented.

The screw 860 is retained in a carriage portion 867 via retaining pins 883 that fit into body detents 884. In an illustrative embodiment, the carriage portion 867 is part of the fixed block 661. Once desired rotation of the screw 860 is achieved, the screw 860 can be locked in place via one or more locking pins 881 that fit into locking detents 880 in the head portion of the screw 860.

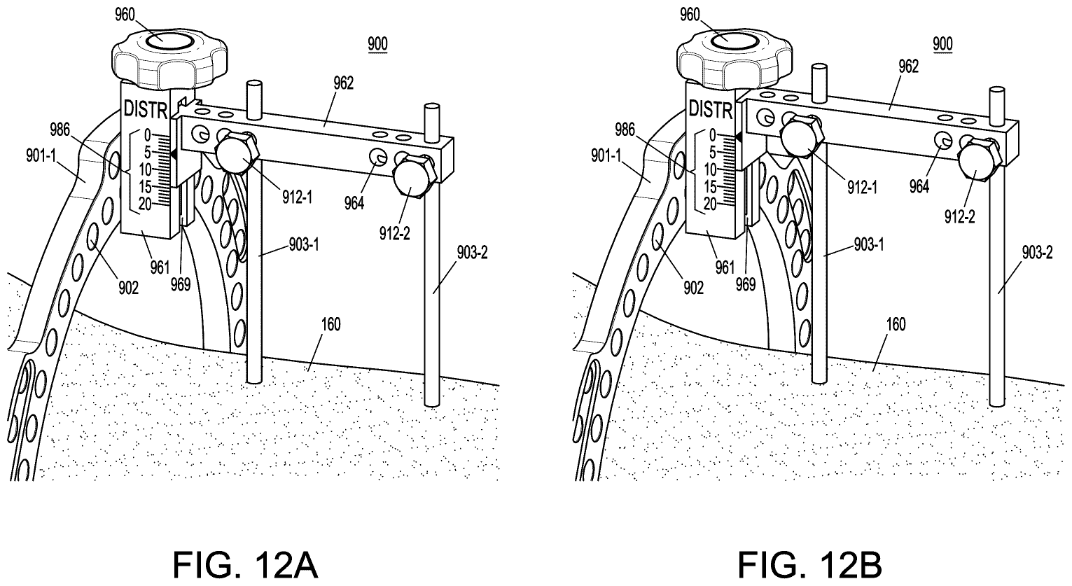

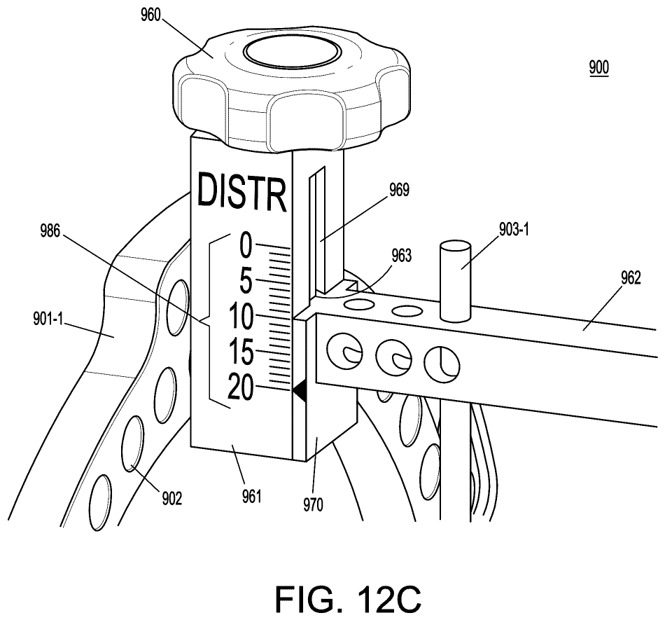

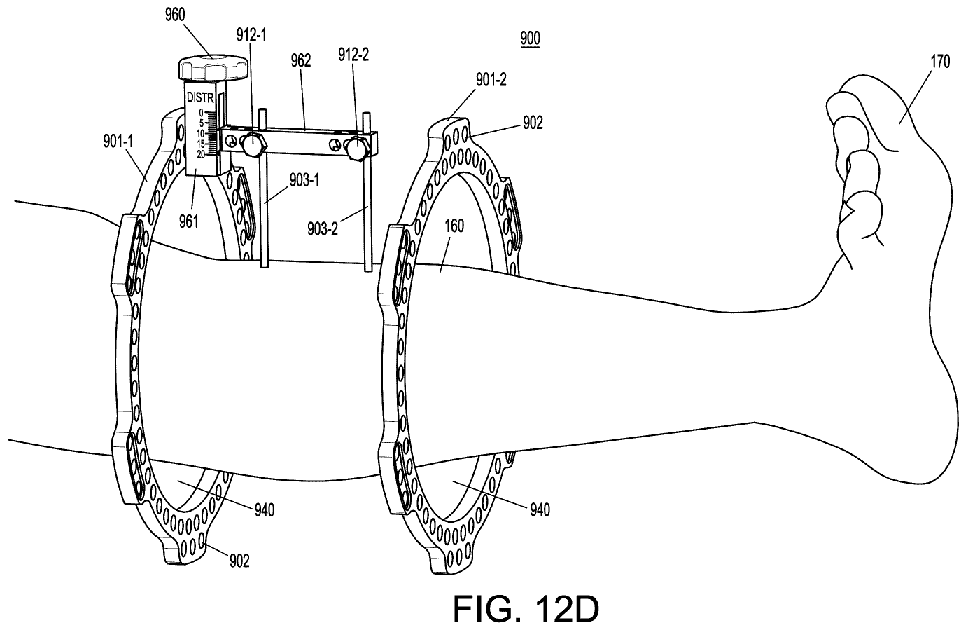

FIGS. 12A and 12B illustrate an external fixation device 900 for translation of a bone segment, in accordance with an alternative embodiment. FIG. 12C illustrates an enlarged view of a portion of the external fixation device 900 of FIGS. 12A and 12B. FIG. 12D illustrates the external fixation device 900 around a leg 160 of a patient in a position relative to a foot 170. Elements the same or similar to those described in connection with previous embodiments are designated by similar reference numbers. Like the external fixation device 100, the external fixation device 900 includes a first external fixation element 901-1 and a second external fixation element 901-2 (collectively, “external fixation elements 901”). In some illustrative embodiments, in connection with the external fixation device 900, the second external fixation element 901-2 can be omitted.

In an illustrative embodiment, the external fixation elements 901 comprise circular-shaped frames. However, in other embodiments, the external fixation elements 901 may be, for example, oval-shaped, square-shaped, rectangular-shaped, or any other regular or irregular shape configured for an external fixation application. The external fixation elements 901 respectively comprise a plurality of fastener apertures 902 through which fasteners such as, for example, bolts or screws may be positioned. The external fixation elements 901 respectively further comprise a central aperture 940 that is configured and sized to receive one or more body structures (e.g., a portion of a leg or arm) therethrough.

Although not shown in FIG. 12D, the first external fixation element 901-1 and the second external fixation element 901-2 can be coupled to each other via one or more connection struts (similar to the connection struts 635 in FIG. 9A) extending between the first and second external fixation elements 901-1 and 902-2. Fasteners such as, for example, bolts or screws may be positioned through the fastener apertures 902 to secure the connection struts. The connection struts themselves may be positioned through the fastener apertures 902 to be secured to the first and second external fixation elements 901-1 and 901-2.

A fixed block 961 is secured to the first external fixation element 901-1 via one or more fasteners inserted through respective fastener apertures 902 in the first external fixation element 901-1 and corresponding respective fastener apertures (not shown) in the fixed block 961. The fixed block 961 is coupled to a translating block 662 via an extension portion 963 extending from a guide plate 970 of the translating block 662. The extension portion 963 is sized and shaped to fit in and slide along a channel 969 of the fixed block 961. In an illustrative embodiment, the extension portion 963 may be the same as or similar to the protruding portion 663, including a threaded hole (not shown) in the extension portion 963 to mate with a screw 960 disposed through a threaded fastener aperture in the fixed block 961. The screw may be similar to the worm screw 660. Similar to the transverse adjustment element 110, rotation of the screw 960, by virtue of its engagement with the extension portion 963 causes the translating block 962 to move in first and second transverse directions (e.g., along the transverse plane) perpendicular to the longitudinal axis and to a longitudinal direction of a bone or the leg 160. For example, turning (rotating) the screw 960 in the clockwise direction while causes the translating block 962 to move in a first transverse direction (e.g., along the transverse plane) away from a leg 160 and a bone of the leg 160. Alternatively, turning (rotating) the screw 960 in the counter-clockwise direction causes the translating block 962 to move in a second (opposite) transverse direction (e.g., along the transverse plane in an opposite direction) toward the leg 160 and a bone of the leg 160. The translating block 962 moves in a direction perpendicular to the longitudinal axis shown in FIG. 4B.

As shown in FIG. 12A, first and second pins 903-1 and 903-2 are attached to the translating block 962. The first and second pins 903-1 and 903-2 are attached to the translating block 962 through respective fasteners 912-1 and 912-2 engaging the first and second pins 903-1 and 903-2 through apertures 964 formed in the translating block 962. For example, the respective apertures 964 and the respective fasteners 912-1 and 912-2 can be threaded so that upon screwing the respective fasteners 912-1 and 912-2 into the apertures 964 and engaging the first and second pins 903-1 and 903-2, the first and second pins 903-1 and 903-2 can be secured in place on the translating block 962. Similar to the first and second pins 103-1 and 103-2 (and 603-1 and 603-2), the first and second pins 903-1 and 903-2 engage and are secured to a bone segment, which is like the bone segment 152 or 652.

As a result, when the translating block 962 is translated, the attached corresponding first and second pins 903-1 and 903-2 and the bone segment move in a direction consistent with the movement of the translating block 962. For example, turning (rotating) the screw 960 in the clockwise direction while mated with the extension portion 963 causes the translating block 962, attached first and second pins 903-1 and 903-2 and the to move in a first transverse direction (e.g., along the transverse plane) away from a bone. Alternatively, turning (rotating) the screw 960 in the counter-clockwise direction while mated with the extension portion 963 causes the translating block 962, attached first and second pins 903-1 and 903-2 and the bone segment to move in a second (opposite) transverse direction (e.g., along the transverse plane in an opposite direction) toward the bone.

In illustrative embodiments, the fixed block 961 includes a numbered indexing mechanism 986 calibrated with the motion of the screw 960 to precisely control the stroke per turn. As can be seen in FIGS. 12A-12D, the guide plate 970 includes an arrow which lines up with scaled distances in a range of, for example, 0 mm to 20 mm. In an illustrative embodiment, a patient may have prescribed movements of the translating block 962. For example, a patient may be prescribed a daily series of movements that start at prescribed increment on the scale (example: 5 mm or 10 mm starting point). Each line on the scale can represent a single day of movement. The screw 960 may be turned one full rotation per day, which, for example, could be performed in 1/3 rotation movements three times a day (e.g., morning, noon and night). Each full rotation (e.g., one complete clockwise rotation) moves the translating block 1 mm farther away from the bone toward 0 mm on the scale from the original set position (e.g., 5 mm or 10 mm in this example). When the 0 mm mark is reached, the patient may be instructed to reverse the movement until the bone segment is returned to its original anatomical position. FIG. 12A shows the translating block 962 at 5 mm and FIG. 12B shows the translating block 962 at 0 mm.

The amount of translation to reach 0 mm can easily be set by loosening the first and second fasteners 912-1 and 912-2 to slide the translating block 962 and connected guide plate 970 closer to the leg 160 with respect to the first and second pins 903-1 and 903-2, which have been fixed to the bone segment. Once the translating block 962 is in the desired position, the first and second fasteners 912-1 and 912-2 can be tightened to fix the translating block 962 to the first and second pins 903-1 and 903-2 at the desired value (e.g., 5 mm, 10 mm, 15 mm, 20 mm). For example, FIG. 12C shows the translating block 962 at 20 mm. Moving the translating block 962 along the scale toward 0 mm by rotating the screw 960 (e.g., rotating the screw 960 clockwise) can commence according to the prescription until 0 mm is reached. Once 0 mm is reached, a prescription can be followed to return the translating block 962, corresponding first and second pins 903-1 and 903-2 and attached bone segment, back to the original starting position (e.g., 5 mm, 10 mm, 15 mm, 20 mm), by turning the screw 960 in the opposite direction (e.g., counterclockwise) according to the prescribed daily amount. Although discussed in terms of millimeters, the embodiments are not limited thereto and other units of measurement can be used.

As noted in connection with FIG. 11, a stroke limiting element 800 can be used to limit the stroke distance S (distance of travel) that a translating block 962 translates. For example, the stroke limiting element 800 or another type of stroke limiting element can be incorporated into the fixed block 961 to stop translation of the bone segment at the 0 mark of the numbered indexing mechanism 986. Accordingly, a screw 960 that actuates the translation may be rotated to create the translation until such a point whereby the screw 960 bottoms out after traveling the designated stroke distance S, thus stopping any further translation (e.g., at 0 mm).

In some embodiments, the screw 960 may include a haptic type of feedback to notify a user of when to stop rotation. The screw 960 may be equipped with a clutching mechanism to prevent overloading or fracturing of a bone segment. In some illustrative embodiments, the screw 960 may be controlled by a programmable stopping mechanism that prevents overtravel that could potentially break through the skin and controls the stroke of the screw 960 as it returns the bone segment to its original anatomical position. The programmable stopping mechanism may be electronically linked to the screw 960. In some embodiments, the programmable stopping mechanism may be remotely controlled such as through an application on a computer or mobile device. The external fixation device 900 is not limited to a screw 960, and may include other mechanisms to effect rotation and corresponding movement of the translating block 962 such as, for example, a motor or switch-operated mechanism similar to the motorized and switch-operated transverse adjustment elements 210 and 310.

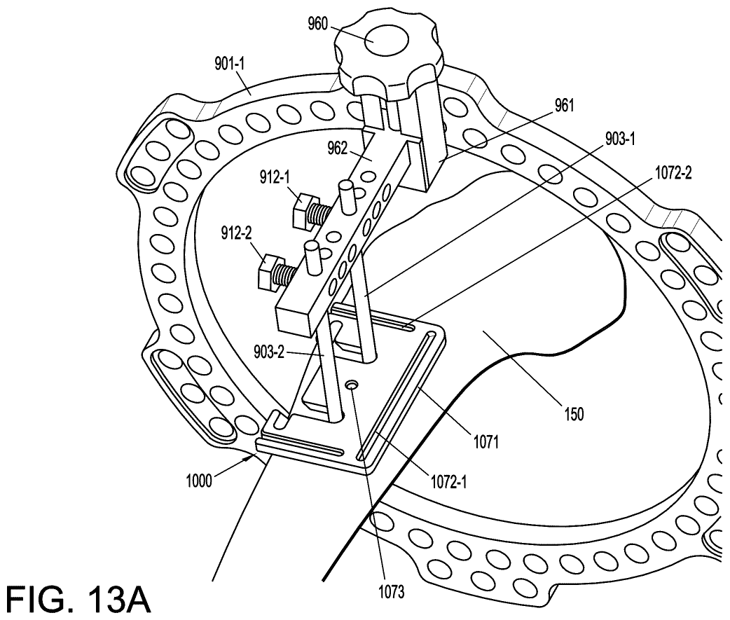

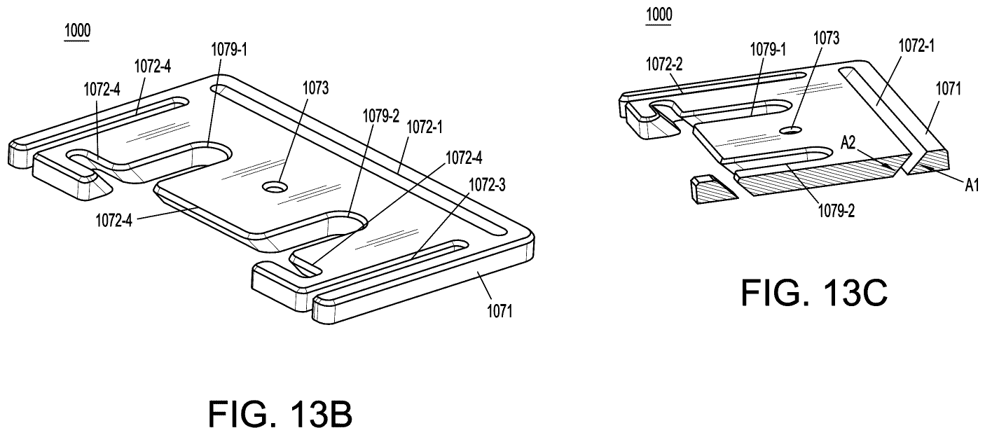

Referring to FIGS. 13A-13C, an alternative embodiment of a corticotomy and pin targeting guide 1000 is shown in conjunction with the external fixation device 900. Referring to FIGS. 13A-13C, a corticotomy and pin targeting guide 1000 assures alignment of first and second pins 903-1 and 903-2 with a corticotomy in a bone (e.g., bone 150). The corticotomy and pin targeting guide 1000 includes a plurality of saw slots 1072-1, 1072-2, 1072-3 and 1072-4 (collectively “saw slots 1072”) formed through a plate 1071 around the perimeter of the plate 1071. The corticotomy and pin targeting guide 1000 further includes a first pin slot 1079-1 corresponding to the first pin 903-1 and a second pin slot 1079-2 corresponding to the second pin 903-2. As can be seen, the first and second pin slots 1079-1 and 1079-2 are each open at one end to permit placement around or removal from pins that are affixed to a bone. The corticotomy and pin targeting guide 1000 further includes at least one K-wire hole 1073. Similar to the K-wire holes 773, the K-wire hole 1073 can be used to align with points on a bone 150 where K-wires need to be inserted into the bone 150 for stabilization of the corticotomy and pin targeting guide 1000 and bone 150 during creation of the cortical window. A K-wire can be inserted through the K-wire hole 1073 into the bone 150 and removed following creation of the cortical window. Although one K-wire hole 1073 is shown, more than one K-wire hole can be used.

The corticotomy and pin targeting guide 1000 aligns the first and second pins 903-1 and 903-2 such that the first and second pins 903-1 and 903-2 can be perfectly placed or substantially perfectly placed in the center of the corticotomy. The saw slots 1072 allow a surgeon to perforate the bone 150 in a perfect or substantially perfect rectangle of a known dimension to create the cortical window. In more detail, the first pin 903-1 of the external fixation device 900 is affixed to the bone 150 by a surgeon. Then, the plate 1071 is placed on the bone 150, with the first pin slot 1079-1 being placed around the first pin 903-1 by engaging the first pin 903-1 via the open end of the first pin slot 1079-1 such that the closed end of the first pin slot 1079-1 rests on the first pin 903-1. Depending on the surgical technique, the plate 1071 may be disposed directly on the bone 150 or with intervening layers (e.g., skin, fat, muscle, etc.) between the plate 1071 and the bone 150.

Following placement of the plate 1071 with the first pin slot 1079-1 being placed around the first pin 903-1, a K-wire is inserted through the K-wire hole 1073 into the bone 150 for additional stabilization. Then, the second pin 903-2 is driven through the second pin slot 1079-2 into the bone 150 so that, like the first pin 903-1, the second pin 903-2 is affixed to the bone 150. Like the first pin 903-1, the closed end of the second pin slot 1079-2 rests on the second pin 903-2. The first and second pin slots 1079-1 and 1079-2 are spaced apart from each other at a desired distance for placement of the first and second pins 903-1 and 903-2 in the bone 150.

Following fixing of the second pin 903-2, a surgeon creates a cortical window with a bone saw (e.g., sagittal saw) using the saw slots 1072 to guide a blade of the bone saw to cut out a bone segment (e.g., like bone segment 152/652) from the bone 150 in a rectangular shape (e.g., cut out length and width) or a square shape. The bone segment (cortical window) is created by cutting the bone with a blade of a bone saw engaging the bone 150 through the saw slots 1072. As can be seen in detail in FIGS. 13B and 13C, each of the slots 1072 includes angled edges where the angles A1 and A2 are, for example, 45 degrees and 135 degrees, respectively. The embodiments are not limited to the noted values for angles A1 and A2 and may have other values less than or greater than the noted values. The function of the angled slots is to create a bone segment with inwardly angled (e.g., mitered) edges such that when the bone segment is returned to its original anatomical position, the bone segment cannot be pushed down further into the bone 150 than its original anatomical position due to the inwardly angled edges, which would prevent recessing the bone segment deeper into the bone 150 than the original anatomical position of the bone segment. Accordingly, by virtue of using the angled saw slots 1072 as guides when cutting the bone 150, the resulting bone segment has a built-in safety mechanism to prevent recessing the bone segment deeper into the bone 150 than its original anatomical position, which may be caused, for example, by a patient over-rotating the screw 960 when returning the bone segment from a translated position away from the bone 150. The angled saw slots 1072 therefore allow for the creation of a bone segment with inwardly angled edges that aid in the return, centering and seating of the bone segment, which can promote healing of the bone segment upon the return to its original anatomical position.

After creation of the cortical window (bone segment), the corticotomy and pin targeting guide 1000 is removed. The removal process includes removing the K-wire from the K-wire hole 1073 and removing the plate 1071 from engagement with the first and second pins 903-1 and 903-2. The plate 1071 is moved so that the closed ends of the first and second pin slots 1079-1 and 1079-2 are moved away from the first and second pins 903-1 and 903-2 and the first and second pins 903-1 and 903-2 exit the first and second pin slots 1079-1 and 1079-2 through the open ends of the first and second pin slots 1079-1 and 1079-2 so that the plate 1071 can be removed.

Multiple sizes of plate 1071, including different lengths of the saw slots 1072 and spacing for the first and second pin slots 1079-1 and 1079-2, are contemplated depending on a desired size of the cortical window and/or the anatomy of a patient and/or the underlying bonc.

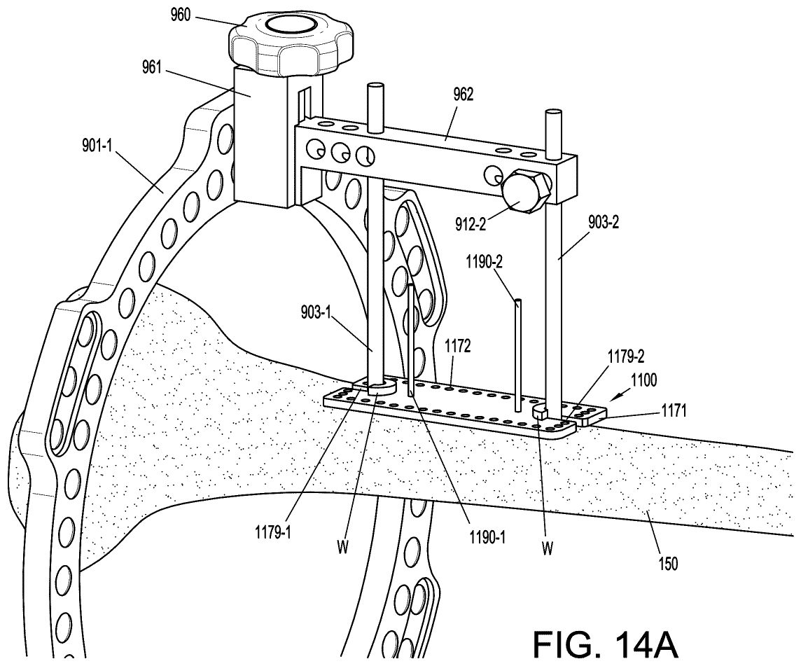

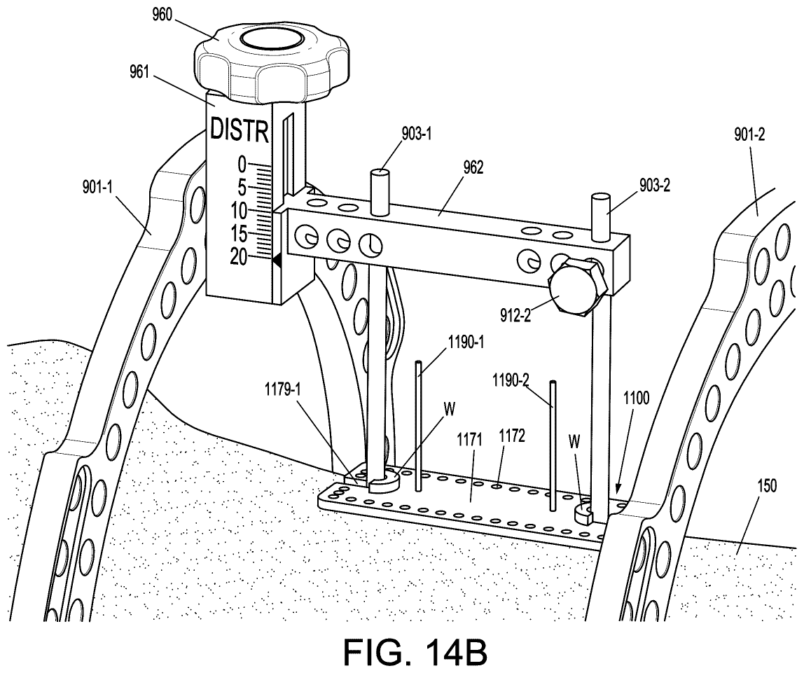

Referring to FIGS. 14A and 14B, another alternative embodiment of a corticotomy and pin targeting guide 1100 is shown in conjunction with the external fixation device 900. Referring to FIGS. 14A and 14B, a corticotomy and pin targeting guide 1100 assures alignment of first and second pins 903-1 and 903-2 with a corticotomy in a bone (e.g., bone 150). The corticotomy and pin targeting guide 1100 includes a plurality of drill holes 1172 through a plate 1171 around the perimeter of the plate 1171. The corticotomy and pin targeting guide 1100 further includes a first pin slot 1179-1 corresponding to the first pin 903-1 and a second pin slot 1179-2 corresponding to the second pin 903-2. As can be seen, the first and second pin slots 1179-1 and 1179-2 are each open at one end to permit placement around or removal from pins that are affixed to a bone. The corticotomy and pin targeting guide 1100 further includes K-wire holes. Similar to the K-wire holes 773 and 1073, the K-wire holes in the plate 1171 can be used to align with points on a bone 150 where first and second K-wires 1190-1 and 1190-2 need to be inserted into the bone 150 for stabilization of the corticotomy and pin targeting guide 1100 and bone 150 during creation of the cortical window. First and second K-wires 1190-1 and 1190-2 can be inserted through the K-wire holes into the bone 150 and removed following creation of the cortical window. Although two K-wire holes for the first and second K-wires 1190-1 and 1190-2 are shown, more or less than two K-wire holes can be used.

The corticotomy and pin targeting guide 1100 aligns the first and second pins 903-1 and 903-2 such that the first and second pins 903-1 and 903-2 can be perfectly placed or substantially perfectly placed in the center of the corticotomy. The drill holes 1172 allow a surgeon to perforate the bone 150 in a perfect or substantially perfect rectangle of a known dimension to create the cortical window. In more detail, the first pin 903-1 of the external fixation device 900 is affixed to the bone 150 by a surgeon. Then, the plate 1171 is placed on the bone 150, with the first pin slot 1179-1 being placed around the first pin 903-1 by engaging the first pin 903-1 via the open end of the first pin slot 1179-1 such that the closed end of the first pin slot 1179-1 rests on the first pin 903-1. Depending on the surgical technique, the plate 1171 may be disposed directly on the bone 150 or with intervening layers (e.g., skin, fat, muscle, etc.) between the plate 1171 and the bone 150.

Following placement of the plate 1171 with the first pin slot 1179-1 being placed around the first pin 903-1, the first and second K-wires 1190-1 and 1190-2 are inserted through the K-wire holes into the bone 150 for additional stabilization. Then, the second pin 903-2 is driven through the second pin slot 1179-2 into the bone 150 so that, like the first pin 903-1, the second pin 903-2 is affixed to the bone 150. Like the first pin 903-1, the closed end of the second pin slot 1179-2 rests on the second pin 903-2. As can be seen in FIGS. 14A and 14B, the closed ends of the first and second pin slots 1179-1 and 1179-2 include raised wall portions W extending perpendicularly from the plate 1171 to provide added support for the first and second pins 903-1 and 903-2. The first and second pin slots 1179-1 and 1179-2 are spaced apart from each other at a desired distance for placement of the first and second pins 903-1 and 903-2 in the bone 150.

Following fixing of the second pin 903-2, a surgeon creates a cortical window with a drill using the drill holes 1172 to create a series of holes in a rectangular or square pattern to cut out a bone segment (e.g., like bone segment 152/652) from the bone 150 in a rectangular or square shape. The drill holes 1172 allow a surgeon to perforate the bone 150 in a perfect or substantially perfect rectangle of a known dimension to create the cortical window. Once the holes are created and the corticotomy and pin targeting guide 1100 is removed, an osteotome can be used to remove (e.g., chisel) remaining bone between the drilled holes and complete the cuts around the bone segment (cortical window).

The removal process of the corticotomy and pin targeting guide 1100 includes removing the first and second K-wires 1190-1 and 1190-2 from the K-wire holes and removing the plate 1171 from engagement with the first and second pins 903-1 and 903-2. The plate 1171 is angled away from the bone 150 and moved so that the closed ends of the first and second pin slots 1179-1 and 1179-2 are moved away from the first and second pins 903-1 and 903-2 and the first and second pins 903-1 and 903-2 exit the first and second pin slots 1179-1 and 1179-2 through the open ends of the first and second pin slots 1179-1 and 1179-2 so that the plate 1171 can be removed.

Multiple sizes of plate 1171, including different numbers of the drill holes 1172 and spacing for the first and second pin slots 1179-1 and 1179-2, are contemplated depending on a desired size of the cortical window and/or the anatomy of a patient and/or the underlying bonc.

One or more components of the bone segment translation systems disclosed herein may be made from, for example: (i) any biocompatible material or treated biocompatible material that can be inserted into soft tissue and/or bones; (ii) a plastic; (iii) a fiber; (iv) a polymer; (v) a metal (e.g., titanium and/or an alloy such as Ti Al Nb, TI-6Al-4V, stainless steel); (vi) a radiolucent material (e.g., carbon fiber, PEEK or aluminum); or (vii) any combination thereof.

According to an illustrative embodiment, a bone segment translation system comprises a first external fixation element, a second external fixation element, a connecting element disposed between and connected to the first external fixation element and the second external fixation element, and a plurality of pins movably coupled to the connecting element.

The first external fixation element and the second external fixation element can respectively comprise at least one of a circular-shaped frame and an oval-shaped frame. A rod can be movably coupled to the connecting element, wherein the plurality of pins are movably coupled to the connecting element via the rod. The rod can be movably coupled to the connecting element by a threaded adjustment element, wherein rotation of the threaded adjustment element causes the rod to move in a direction perpendicular to a surface of the threaded adjustment element. Clockwise rotation of the threaded adjustment element may cause the rod to move in a first direction perpendicular to the surface of the threaded adjustment element, and counter-clockwise rotation of the threaded adjustment element may cause the rod to move in a second direction perpendicular to the surface of the threaded adjustment element, wherein the second direction is opposite the first direction. The threaded adjustment element may comprise a motor to rotate the threaded adjustment element.

The rod can be movably coupled to the connecting element by a switch assembly comprising a switch element, wherein toggling of the switch element causes the rod to move in a direction perpendicular to a surface of the switch assembly. The bone segment translation system may further comprise a pin holder to which the plurality of pins are mounted, wherein the pin holder is coupled to the rod. The plurality of pins can be configured to be affixed to a bone segment.

One of the first external fixation element and the second external fixation element can be coupled to a footplate, wherein the footplate is coupled to one or more pins, the one or pins being affixed to a foot of a subject.

According to an illustrative embodiment, a method for transverse translation of a bone segment with respect to a bone comprises fixing a first external fixation element around a limb of a subject, wherein the limb comprises the bone, fixing a second external fixation element around the limb of the subject, wherein the first external fixation element is spaced apart from the second external fixation element. The method further comprises connecting a connecting element between the first external fixation element and the second external fixation element, and affixing a plurality of pins to the bone segment, wherein the plurality of pins are movably coupled to the connecting element.

The plurality of pins can be movably coupled to the connecting element via a rod movably coupled to the connecting element, wherein the rod is movably coupled to the connecting element by a threaded adjustment element. In illustrative embodiments, rotating the threaded adjustment element causes the rod, the plurality of pins and the bone segment to move in a transverse direction with respect to the bone. Rotating the threaded adjustment element clockwise can cause the rod, the plurality of pins and the bone segment to move in a first transverse direction away from the bone, and rotating the threaded adjustment element counter-clockwise can cause the rod, the plurality of pins and the bone segment to move in a second transverse direction toward the bone.

The rod can be movably coupled to the connecting element by a switch assembly comprising a switch element. Toggling the switch element can cause the rod, the plurality of pins and the bone segment to move in a transverse direction with respect to the bone.

According to an illustrative embodiment, an external fixation device comprises a first external fixation element, a second external fixation element, and a plurality of pins coupled to a moveable rod disposed between the first external fixation element and the second external fixation element.

A connecting element may be disposed between and connected to the first external fixation element and the second external fixation element, wherein the moveable rod is movably coupled to the connecting element.

According to an illustrative embodiment, a bone segment translation system comprises a first external fixation element and a second external fixation element. The first external fixation element and the second external fixation element are coupled to each other through one or more struts disposed between and connected to the first external fixation element and the second external fixation element. A first block is fixed to one of the first external fixation element and the second external fixation element and a second block is movably coupled to the first block. A plurality of pins are coupled to the second block.

The plurality of pins can be configured to be affixed to a bone segment of a bone of a subject. The bone segment translation system may further comprise a targeting guide to create the bone segment and to align the plurality of pins with the bone segment. The targeting guide may comprise a plurality of open-ended slots, wherein respective ones of the plurality of open-ended slots engage respective ones of the plurality of pins. The targeting guide may comprise a plurality of angled slots configured for receiving a blade of a bone saw to cut out the bone segment from the bone. The second block can be movably coupled to the first block by a threaded adjustment element, wherein rotation of the threaded adjustment element causes the second block to move in a direction perpendicular to a surface of the bone. The bone segment translation system may further comprise a stroke limiting element, wherein rotation of the threaded adjustment element is limited by the stroke limiting element.

According to an illustrative embodiment, a bone segment translation system comprises an external fixation element, a pin supporting element movably coupled to the external fixation element, and a plurality of pins connected to the pin supporting element. The external fixation element comprises at least one of a circular-shaped frame and an oval-shaped frame. The plurality of pins may be configured to be affixed to a bone segment of a bone of a subject.

According to an illustrative embodiment, a bone segment translation system comprises an external fixation element, a first block fixed to the external fixation element, a second block movably coupled to the first block, and a plurality of pins coupled to the second block.

The plurality of pins may be configured to be affixed to a bone segment of a bone of a subject. The bone segment translation system further comprises a targeting guide that can be used in connection with creating the bone segment and aligning the plurality of pins with the bone segment. The targeting guide may comprise a plurality of open-ended slots, wherein respective ones of the plurality of open-ended slots engage respective ones of the plurality of pins. The targeting guide may comprise a plurality of angled slots configured for receiving a blade of a bone saw to cut out the bone segment from the bone.

The second block may be movably coupled to the first block by a threaded adjustment element, wherein rotation of the threaded adjustment element causes the second block to move in a transverse direction with respect to the bone. The rotation of the threaded adjustment element in a first rotational direction causes the second block to move in a first transverse direction with respect to the bone, and the rotation of the threaded adjustment element in a second rotational direction causes the second block to move in a second transverse direction with respect to the bone, wherein the second transverse direction is opposite the first transverse direction.

The bone segment translation system may further comprise a stroke limiting element to limit the rotation of the threaded adjustment element. The stroke limiting element may comprise a floor that prevents the rotation of the threaded adjustment element beyond a designated distance.

The bone segment translation system may further comprise an additional external fixation element, wherein the external fixation element and the additional external fixation element are coupled to each other through one or more struts disposed between and connected to the external fixation element and the additional external fixation element. The external fixation element may comprise at least one of a circular-shaped frame and an oval-shaped frame.

According to an illustrative embodiment, a bone segment translation system comprises an external fixation element, a pin supporting element movably coupled to the external fixation element, and a plurality of pins connected to the pin supporting element.

The pin supporting element can be movably coupled to the external fixation element via a fixed block coupled to the external fixation element. The bone segment translation system may further comprise a threaded adjustment element disposed in the fixed block and engaged with the pin supporting element. The plurality of pins can be configured to be affixed to a bone segment of a bone of a subject, and rotation of the threaded adjustment element can cause the pin supporting element to move in a transverse direction with respect to the bone. The external fixation element can comprise at least one of a circular-shaped frame and an oval-shaped frame.

According to an illustrative embodiment, a bone segment translation system comprises a frame disposed around a bone of a subject, a first block fixed to the frame, a second block movably coupled to the first block, and one or more pins coupled to the second block.

The one or more pins can be configured to be affixed to a bone segment of the bone, and the second block may be movable in a transverse direction with respect to the bone. The bone segment translation may further comprise a threaded adjustment element disposed in the second block and engaged with the second block, wherein rotation of the threaded adjustment element causes the second block to move in the transverse direction with respect to the bone.

As noted above, current approaches fail to effectively implement bone segment translation by failing to sufficiently deliver important nutrients to affected areas of the body. In addition, current techniques rely on configurations that are not structurally sound, and may not be able to support the weight of a patient that has already been destabilized and potentially weakened by bone segment creation and translation. As a result, current approaches could become associated with major complications that could result in a failed procedure or even lead to amputation. Advantageously, the illustrative embodiments provide a bone segment translation system that addresses the inherent limitations and structural shortcomings of conventional approaches. For example, one or more illustrative embodiments include a member that is fixed to a rigid and strong circular (or substantially circular) fixator (e.g., external fixation element) where a fixation element-to-fixation element construct with, for example, struts spanning between the fixation elements (e.g., ring-shaped fixation elements) can fully carry the load of patient's weight while ambulating on a leg receiving transverse translational therapy. Illustrative embodiments advantageously protect a limb in treatment from potentially fracturing while simultaneously providing a device to translate a bone segment to achieve a therapeutic goal of revascularization.

As an additional advantage, the illustrative embodiments allow for the treatment of ischemic lower legs and feet where, in addition to the translation procedure, other procedures can be performed to remove necrotic (dead) tissue, place skin graft materials in affected areas and fix deformities in affected areas. The bone segment translation system in illustrative embodiments is compatible with multiple components designed to protect the foot. In addition, the use of the external fixation elements allows for device configurations that can make corrections to foot deformities while simultaneously implementing bone translation and providing necessary structural support to weakened areas to prevent catastrophic leg fractures. Accordingly, the embodiments provide for increased safety and versatility when compared with current techniques.

Although exemplary embodiments of the present invention have been described hereinabove, it should be understood that the present invention is not limited to these embodiments but may be modified by those skilled in the art without departing from the spirit and scope of the present invention. For example, the functionality described herein may be applied to other embodiments as enhancements or improvements as the design becomes progressively elaborative.

What is claimed is:

1. A bone segment translation system comprising:
an external fixation element;
a first block fixed to the external fixation element;
a second block movably coupled to the first block; and
a plurality of pins coupled to the second block.

2. The bone segment translation system of claim 1, wherein the plurality of pins are configured to be affixed to a bone segment of a bone of a subject.

3. The bone segment translation system of claim 2, further comprising a targeting guide used in connection with creating the bone segment and aligning the plurality of pins with the bone segment.

4. The bone segment translation system of claim 3, wherein the targeting guide comprises a plurality of open-ended slots, wherein respective ones of the plurality of open-ended slots engage respective ones of the plurality of pins.

5. The bone segment translation system of claim 3, wherein the targeting guide comprises a plurality of angled slots configured for receiving a blade of a bone saw to cut out the bone segment from the bone.

6. The bone segment translation system of claim 2, wherein the second block is movably coupled to the first block by a threaded adjustment element.

7. The bone segment translation system of claim 6, wherein rotation of the threaded adjustment element causes the second block to move in a transverse direction with respect to the bone.

8. The bone segment translation system of claim 7, wherein:
the rotation of the threaded adjustment element in a first rotational direction causes the second block to move in a first transverse direction with respect to the bone;
the rotation of the threaded adjustment element in a second rotational direction causes the second block to move in a second transverse direction with respect to the bone; and
the second transverse direction is opposite the first transverse direction.

9. The bone segment translation system of claim 7, further comprising a stroke limiting element, wherein the rotation of the threaded adjustment element is limited by the stroke limiting element.

10. The bone segment translation system of claim 9, wherein the stroke limiting element comprises a floor that prevents the rotation of the threaded adjustment element beyond a designated distance.

11. The bone segment translation system of claim 1, further comprising an additional external fixation element, wherein the external fixation element and the additional external fixation element are coupled to each other through one or more struts disposed between and connected to the external fixation element and the additional external fixation element.

12. The bone segment translation system of claim 1, wherein the external fixation element comprises at least one of a circular-shaped frame and an oval-shaped frame.

13. A bone segment translation system comprising:
an external fixation element;
a pin supporting element movably coupled to the external fixation element; and
a plurality of pins connected to the pin supporting element.

14. The bone segment translation system of claim 13, wherein the pin supporting element is movably coupled to the external fixation element via a fixed block coupled to the external fixation element.

15. The bone segment translation system of claim 14, further comprising a threaded adjustment element disposed in the fixed block and engaged with the pin supporting element.

16. The bone segment translation system of claim 15, wherein:
the plurality of pins are configured to be affixed to a bone segment of a bone of a subject; and
rotation of the threaded adjustment element causes the pin supporting element to move in a transverse direction with respect to the bone.

17. The bone segment translation system of claim 13, wherein the external fixation element comprises at least one of a circular-shaped frame and an oval-shaped frame.

18. A bone segment translation system comprising:
a frame disposed around a bone of a subject;
a first block fixed to the frame;
a second block movably coupled to the first block; and
one or more pins coupled to the second block.

19. The bone segment translation system of claim 18, wherein:
the one or more pins are configured to be affixed to a bone segment of the bone; and
the second block is movable in a transverse direction with respect to the bone.

20. The bone segment translation system of claim 19, further comprising a threaded adjustment element disposed in the second block and engaged with the second block, wherein rotation of the threaded adjustment element causes the second block to move in the transverse direction with respect to the bone.
